(19) United States
(12) Patent Application Publication
(10) Pub. No.: US 2012/0318317 A1
(43) Pub. Date: Dec. 20, 2012

(54) MOLECULAR THERMOELECTRIC DEVICE

(75) Inventors: Charles A. Stafford, Tucson, AZ (US); Justin P. Bergfield, Tucson, AZ (US)

(73) Assignee: Arizona Board of Regents on behalf of the University of Arizona, Tucson, AZ (US)

(21) Appl. No.: 13/574,835

(22) PCT Filed: Feb. 10, 2011
(86) PCT No.: PCT/US11/24359

§ 371 Date: Sep. 4, 2012

Related U.S. Application Data
(60) Provisional application No. 61/337,660, filed on Feb. 10, 2010.

(51) Int. Cl. H01L 35/24; C07C 321/26; C07C 323/20; H01L 35/28

(52) U.S. Cl. 136/203; 136/205; 568/66; 568/62

(57) ABSTRACT

An enormous order-dependent quantum enhancement of thermoelectric effects in the vicinity of higher-order interferences has been discovered in the transmission spectrum of nanoscale junctions. Significant enhancements due to both transmission nodes and resonances across such junctions are exemplified by single-molecule junctions (SMJs) based on 3,3′-biphenyl and polyphenyl ether (PPE). Thermoelectric devices employing such SMJs offer superior efficiency and performance. Moreover, the enhanced thermoelectric response is not limited to only SMJs, but may be obtained from any junction exhibiting transmission nodes or resonances arising from coherent electronic transport.

### CROSS-REFERENCE TO RELATED APPLICATIONS

This application claims the benefit of priority to U.S. Provisional Application 61/337,660 filed Feb. 10, 2010, which is hereby incorporated by reference herein.

### BACKGROUND

Thermoelectric (“TE”) devices are highly desirable since they can directly convert between thermal and electrical energy. Electrical power can be supplied to such a device to either heat or cool adjoining reservoirs (e.g., Peltier effect) or alternatively, the flow of heat (e.g., waste heat from a factory or automobile) can be converted into usable electrical power (e.g., Seebeck effect). Often, the efficiency of a TE device is characterized by the dimensionless figure-of-merit ZT=S²GT/κ, constructed with the rationale that an efficient TE device should simultaneously: maximize the electrical conductance G so that current can flow without much Joule heating, minimize the thermal conductance K in order to maintain a temperature gradient across the device, and maximize the Seebeck coefficient S to ensure that the coupling between the electronic and thermal currents is as large as possible (Bell, L. E., Cooling, Heating, Generating Power, and Recovering Waste Heat with Thermoelectric Systems. Science 2008, 321, 1457-1461, hereinafter, Bell2008; DiSalvo, F. J., Thermoelectric Cooling and Power Generation. Science 1999, 285, 703-706, hereinafter DiSalvo1999). Generally, however, ZT is difficult to maximize because these properties are highly correlated with one another (Hochbaum, A. I., Chen, R., Delgado, R. D., Liang, W., Garnett, E. C., Najarian, M., Majumdar, A., and Yang, P., Enhanced thermoelectric performance of rough silicon nanowires, Nature 2008, 451, 163-167, hereinafer Hochbaum2008; Majumdar, A., MATERIALS SCIENCE: Enhanced: Thermoelectricity in Semiconductor Nanostructures. Science 2004, 303, 777-778, hereinafter Majumdar2004; Snyder, G. J.; Toberer, E. S. Complex thermoelectric materials. Nat Mater 2008, 7, 105-114, hereinafter Snyder 2008), an effect that can become more pronounced at the nanoscale where the number of degrees of freedom available is small.

If a TE material were found exhibiting ZT it would constitute a commercially viable solution for many heating and cooling problems at both the macro- and nano-scales, with no operational carbon footprint (DiSalvo1999). Currently, the best TE materials available in the laboratory exhibit ZT≈3, whereas for commercially available TE devices ZT≈1, owing to various packaging and fabrication challenges (Bell 2008; Harman, T. C., Taylor, P. J., Walsh, M. P., and LaForge, B. E., Quantum Dot Superlattice Thermoelectric Materials and Devices, Science 2002, 297, 2229-2232, hereinafter Harman 2002).

### Overview

The inventors have discovered that enhanced thermoelectric effects can be found in the vicinity of a transmission node of a quantum tunneling device. Noteably, in some such devices, the transmission probability vanishes quadratically as a function of energy at such a transmission node. Even more significantly, the inventors have discovered that two-terminal Single-Molecule Junctions (“SMJ”s) can also exhibit higher-order “supernodes” in their transmission spectra.

In the vicinity of a 2n^{th} order supernode the transmission probability T(E) for an electron of energy E to tunnel across a junction is given by:

T(E)∝(E−μ_{node})²ⁿ, (1)

where μ_{node} is the energy of the node. The inventors have discovered that junctions possessing such supernodes exhibit a scalable order-dependent quantum-enhanced thermoelectric response. These results are valid for any device with transmission nodes arising from coherent electronic transport. Moveover, in addition to higher-order destructive interferences, the inventors have discovered that higher-order constructive interferences also strongly enhance thermoelectric effects, so that devices with transmission resonances arising from coherent electronic transport also exhibit this highly desirable behavior. The inventors have further devised example embodiments of devices that operate according to this advantageous behavior they discovered.

Hence, in one respect, various embodiments of the present invention provide a thermoelectric device, comprising: a first electrode; a second electrode; and an electrical transmission medium electrically connected to the first and second electrodes, wherein the electrical transmission medium comprises a quantum conductor that exhibits at least one transmission node or transmission resonance due to quantum interference.

In another respect, various embodiments of the present invention provide a thermoelectric power generator for generating a voltage difference between a first electrical contact and a second electrical contact in response to a temperature difference between a first heat-transfer surface and a second heat-transfer surface, the thermoelectric power generator comprising: at least one N-type thermoelectric structure comprising N-type organic molecules arranged in a self-assembled monolayer; and at least one P-type thermoelectric structure comprising P-type organic molecules arranged in a self-assembled monolayer, wherein the at least one N-type thermoelectric structure and the at least one P-type thermoelectric structure are electrically connected in series between the first and second electrical contacts and thermally connected in parallel between the first and second heat-transfer surfaces.

In still another respect, various embodiments of the present invention provide a Peltier cooler for transferring heat from a low-temperature surface to a high-temperature surface in response to an applied voltage between a first electrical contact and a second electrical contact, the Peltier cooler comprising: at least one N-type thermoelectric structure comprising N-type organic molecules arranged in a self-assembled monolayer; and at least one P-type thermoelectric structure comprising P-type organic molecules arranged in a self-assembled monolayer, wherein the at least one N-type thermoelectric structure and the at least one P-type thermoelectric structure are electrically connected in series between the first and second electrical contacts and thermally connected in parallel between the low-temperature and high-temperature surfaces.

In yet another respect, various embodiments of the present invention provide compound of the formula,

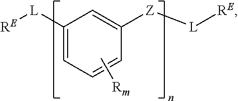

wherein n is 1-100; Z is a bond, —O—, —S—, —N(R^{Z})—, —C(O)—, —S(O)—, —S(O)₂—, —C(R^{Z})₂—, —C(R^{Z})═C(R^{Z})—, —C≡C—, wherein each R^{Z} is independently hydrogen or C₁-C₆ alkyl; each m is independently 0, 1, 2, 3, or 4; each R is independently an electron-donating group, an electron-withdrawing group, or a group electrically similar to hydrogen; each L is independently a bond or a divalent linking group; each R^{E} is independently a functional group capable of bonding to or associating with a metal surface.

In still another respect, various embodiments of the present invention provide an assembly comprising a first metal surface; a second metal surface; and one or more molecules bridging the first and second metal surfaces, wherein each molecule is of the formula

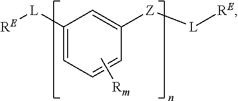

wherein n is 1-100; Z is a bond, —O—, —S—, —N(R^{Z})—, —C(O)—, —S(O)—, —S(O)₂—, —C(R^{Z})_{z}—, —C(R^{Z})═C(R^{Z})—, —C≡C—, wherein each R^{Z} is independently hydrogen or C₁-C₆ alkyl; each m is independently 0, 1, 2, 3, or 4; each R is independently an electron-donating group, an electron-withdrawing group, or a group electrically similar to hydrogen; each L is independently a bond or a divalent linking group; each R^{E} is independently a functional group capable of bonding to or associating with the first metal surface or second metal surface; and wherein for each molecule bridging the first metal surface and second metal surface, one R^{E} group of the molecule is chemically bonded or associated with the first metal surface, and the second R^{E} group of the molecule is chemically bonded or associated with the second metal surface.

These as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 illustrates enhanced thermoelectric response near a 2n^{th} order supernode.

FIG. 2 is a schematic illustration of an example thermoelectric device.

FIG. 3 illustrates thermoelectric characteristics of an example device based on a two-terminal 1,3-benzene Single-Molecule Junction, as determined from many-body theory (a) and Hückel theory (b).

FIG. 4 illustrates: (a) figure of merit ZT, (b) efficiency η, and (c) power P, in the vicinity of a transmission node of an example meta-benzene Single-Molecule Junction, as determined from many-body theory (i) and Hückel theory (ii).

FIG. 5 illustrates a magnified view of figure of merit ZT and efficiency η near a quartic supernode of a 3,3′-biphenyl Single-Molecule Junction.

FIG. 6 illustrates an example of supernode enhancement of ZT, thermopower S and Lorenz number L for polyphenyl ether (PPE) Single-Molecule Junctions with n repeated phenyl groups.

FIG. 7 illustrates transmission probability T(E) and ZT for a 3,3′-biphenyl Single-Molecule Junction with several different phonon transmission values.

FIG. 8 illustrates transmission and ZT in the vicinity of a transmission peak for a tetraphenyl ether (n=4) molecule.

FIG. 9 illustrates an example molecular thermoelectric device incorporating supernode-possessing Single-Molecule Junctions between two electrodes in contact with respective heat reservoirs at different respective temperatures.

FIG. 10 illustrates an example molecular thermoelectric power generator incorporating supernode-possessing Single-Molecule Junctions between a first and a second electrode and between the second and a third electrode, wherein power is generated between the first and third electrodes in response to heat transferred from a cooled reservoir in contact with the second electrode to a heated reservoir in contact with the first and third electrodes.

FIG. 11 illustrates an example molecular Peltier cooler incorporating supernode-possessing Single-Molecule Junctions between a first and a second electrode and between the second and a third electrode, wherein heat is transferred from a cooled reservoir in contact with the second electrode to a heated reservoir in contact with the first and third electrodes in response to a voltage applied between the first and third electrodes.

FIG. 12 illustrates an alternative configuration of the example Peltier cooler of FIG. 11, in which heat is transferred from a cooled reservoir in contact with the first and third electrodes to a heated reservoir in contact with the third electrode in response to a voltage applied between the first and third electrodes.

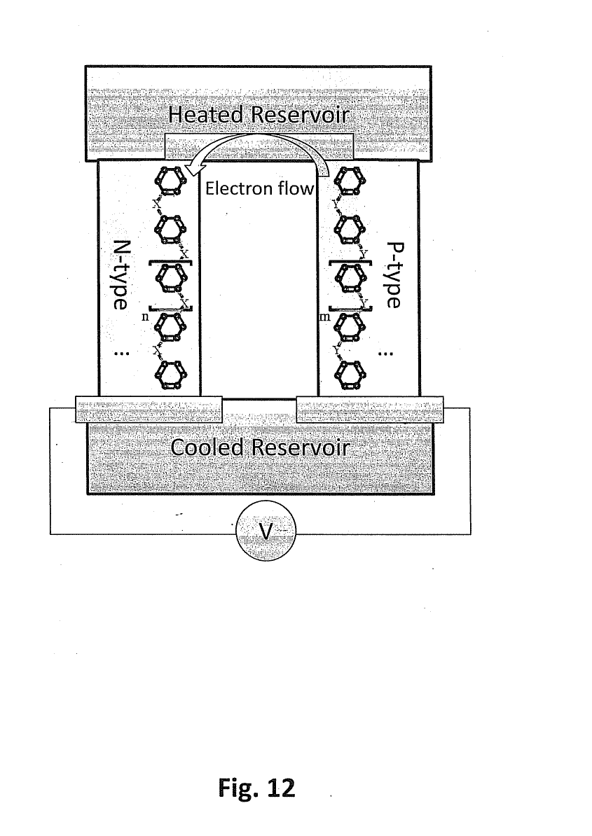

### DETAILED DESCRIPTION

The example embodiments disclosed herein are based, by way of example, on one or another form of Single-Molecule Junction (“SMJ”). As will be described, appropriately constructed molecules can give rise to supernodes as well as transmission resonances. Accordingly, analysis of such molecules serves to illustrate the physical principles underlying enhanced thermoelectric effects on the nanoscale, as well as to provide a framework for fabricating devices that utilize those principles. However, although the focus herein is on molecular junctions, it should be stressed that the results are applicable to any device with transmission nodes or transmission resonances arising from coherent electronic transport. More specifically, any quantum conductor may exhibit transmission nodes or resonances due to quantum interference. Without limitation, examples include semiconductor nanostructures, such as quantum dots and quantum wires, carbon nanotube junctions, and metal nanowires. It should be understood, therefore, that the example embodiments disclosed herein are not limited to molecular junctions.

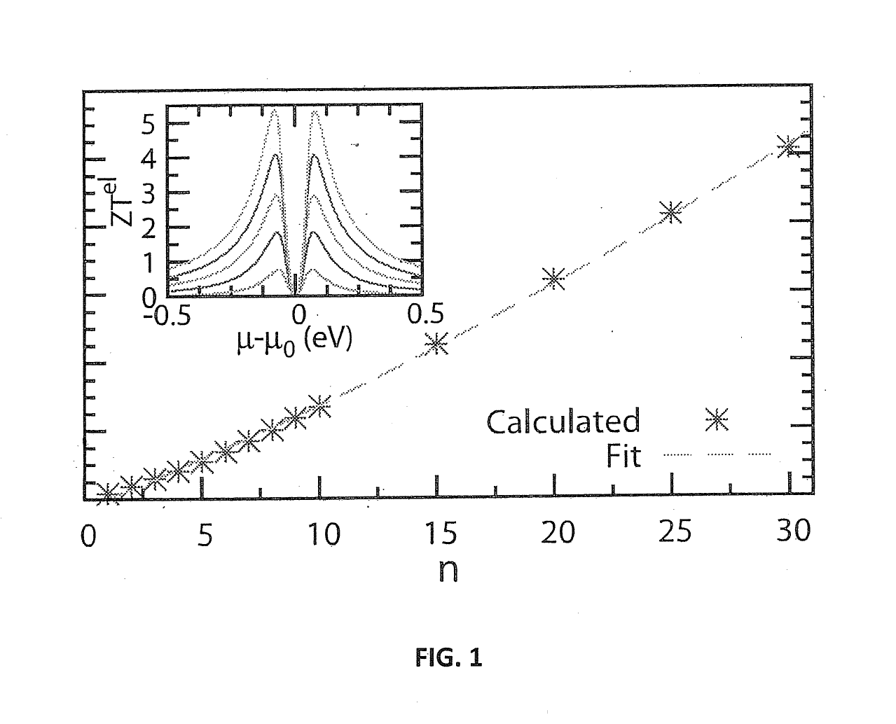

As an example, ZT of a supernode-possessing polyphenyl ether (PPE)-based SMJ is shown as a function of repeated phenyl unit number n in FIG. 1. Based on physical principles discussed below, calculations were performed for a polyphenyl ether (PPE) SMJ with n repeated phenyl groups at room temperature (T=300K) with Γ=0.5 eV. As illustrated in the figure, the maximum value of ZT^{el}, the figure of merit for purely electronic transport, scales super-linearly in n whereby max{ZT^{el}}=4.1 in a junction composed of just four phenyl groups (n=4). More specifically, near a 2n^{th} order supernode in a device's transmission spectrum, we find an order-dependent enhancement of the electronic thermoelectric response potentially limited only by the electronic coherence length. It is evident from FIG. 1 that the enhancement is super-linear in n. Note that the inset in FIG. 1 shows ZT as a function μ for n=1, . . . , 5.

As an engineering rule-of-thumb, ZT has been widely used to characterize the bulk thermoelectric response of materials (Bell 2008; DiSalvo 1999; Snyder 2008). At the nanoscale, however, it is unclear the extent to which ZT is applicable, since bulk scaling relations for transport may break down due to quantum effects (Datta, S. In Electronic Transport in Mesoscopic Systems, Cambridge University Press: Cambridge, UK, 1995, pp 117-174, hereinafter Datta 1995). Moreover, ZT is a linear response metric, and cannot a priori predict nonequilibrium thermoelectric response.

We investigated the efficacy of ZT as a predictor of nonequilibrium device performance at the nanoscale by calculating the thermodynamic efficiency and power of an interacting quantum system using both nonequilibrium many-body theory, following the formalism of [Bergfield, J. P.; Stafford, C. A. Many-body theory of electronic transport in single-molecule heterojunctions. Phys. Rev. B 2009, 79, 245125, hereinafter Bergfield2009a, and incorporated in its entirety herein by reference], and Hückel theory. We discovered that in both theories, variations of ZT and thermodynamic efficiency are in good qualitative agreement. However, significant discrepancies between thermoelectric effects calculated within many-body and Hückel theory are found in the resonant tunneling regime, indicating the essential role of electron-electron interactions in nanoscale thermoelectricity. For a thermoelectric quantum tunneling device, we determined that the power output can be changed significantly by varying an external parameter, such as a gate voltage, and that this variation is not correlated with the variation of ZT. In the next subsection the theoretical foundations of enhanced thermoelectric effects on the nanoscale are presented in more detail.

### 1. Theoretical Foundations of Enhanced Thermoelectric Effect

Neglecting inelastic processes, which are strongly suppressed at room temperature in SMJs, the current flowing into lead 1 of a two-terminal junction may be written as follows (Bergfield, J. P.; Stafford, C. A. Thermoelectric Signatures of Coherent Transport in Single-Molecule Heterojunctions. Nano Letters 2009, 9, 3072-3076, hereinafter Bergfield2009b, and incorporated in its entirety herein by reference):

$${I}_{1}^{( v )} = \frac{1}{h} {\int }_{- ∞}^{∞} d {E ( E - {μ}_{1} )}^{v} T ( E ) [ {f}_{2} ( E ) - {f}_{1} ( E ) ] , \qquad ( 2 )$$

where v=0 (v=1) for the number (heat) current, f_{α} (E) is the Fermi function for lead α with chemical potential μ_{α} and inverse temperature β_{α}, and T(E) is the transmission probability for an electron of energy E to tunnel across the junction. This transmission function may be expressed in terms of the junction's Green's functions as (Datta1995):

T(E)=Tr{Γ¹(E)G(E)Γ²(E)G^{†}(E)}, (3)

where Γ^{α}(E) is the tunneling-width matrix for lead α and G(E) is the retarded Green's function of the SMJ.

In organic molecules, such as those considered herein, electron-phonon coupling is weak, allowing ZT to be expressed as follows:

$$ZT = {ZT}^{el} ( \frac{1}{1 + {κ}^{p h} / {κ}^{el}} ) , \qquad ( 4 )$$

where (Finch, C. M., Garca-Suárez, V. M., Lambert, C. J. Giant thermopower and figure of merit in single-molecule devices. Phys. Rev. B 2009, 79, 033405, hereinafter Finch2009):

$$\begin{matrix}{ZT}^{el} = {( \frac{{L}^{( 0 )} {L}^{( 2 )}}{{[ {L}^{( 1 )} ]}^{2}} - 1 )}^{- 1} \\ and & ( 5 ) \\ {L}^{( v )} ( μ ) = \frac{1}{h} \int d {E ( E - μ )}^{v} T ( E ) ( - \frac{\partial {f}_{0}}{\partial E} ) . & ( 6 )\end{matrix}$$

Here f₀ is the equilibrium Fermi function and κ^{ph}=κ₀T^{ph} is the phonon's thermal conductance, where κ₀=(π²/3)(k_{B}²T/h) is the thermal conductance quantum (Rego, L. G. C., Kirczenow, G., Fractional exclusion statistics and the universal quantum of thermal conductance: A unifying approach. Phys. Rev. B 1999, 59, 13080-13086, hereinafter Rego1999), and T^{ph} is the phonon transmission probability. The electronic thermal conductance is given by:

$${κ}^{el} = \frac{1}{T} ( {L}^{( 2 )} - \frac{{[ {L}^{( 1 )} ]}^{2}}{{L}^{( 0 )}} ) , \qquad ( 7 )$$

where T is the temperature.

The phonon thermal conductance of the junction is typically limited by the lead-molecule interface (Wang, Z., Carter, J. A., Lagutchev, A., Koh, Y. K., Seong, N., Cahill, D. G., and Dlott, D. D., Ultrafast Flash Thermal Conductance of Molecular Chains. Science 2007, 317, 787-790, hereinafter Wang2007). Since the Debye frequency in the metal lead is typically smaller than the lowest vibrational mode of a small organic molecule, the spectral overlap of phonon modes between the two is small, implying T^{ph}=1, so that κ^{ph}=κ₀. Nonetheless, it is claimed that κ^{ph} can reach values as large as 10⁻¹° W/K for some SMJs (Wang 2007; Segal, D., Nitzan, A., and Hänggi, P., Thermal conductance through molecular wires. J. Chem. Phys. 2003, 119, 6840-6855, hereinafter Segal2003), so the correction to ZT due to phonon heat transport (cf., Eq 4) must be taken into account for quantitative estimates of device performance (Liu, Y.-S., Chen, Y.-R., and Chen, Y.-C., Thermoelectric Efficiency in Nanojunctions: A Comparison between Atomic Junctions and Molecular Junctions. ACS Nano 2009, 3, 3497-3504, hereinafter Liu2009). In the following, purely electronic transport is first considered; the effect of phonons is discussed subsequently.

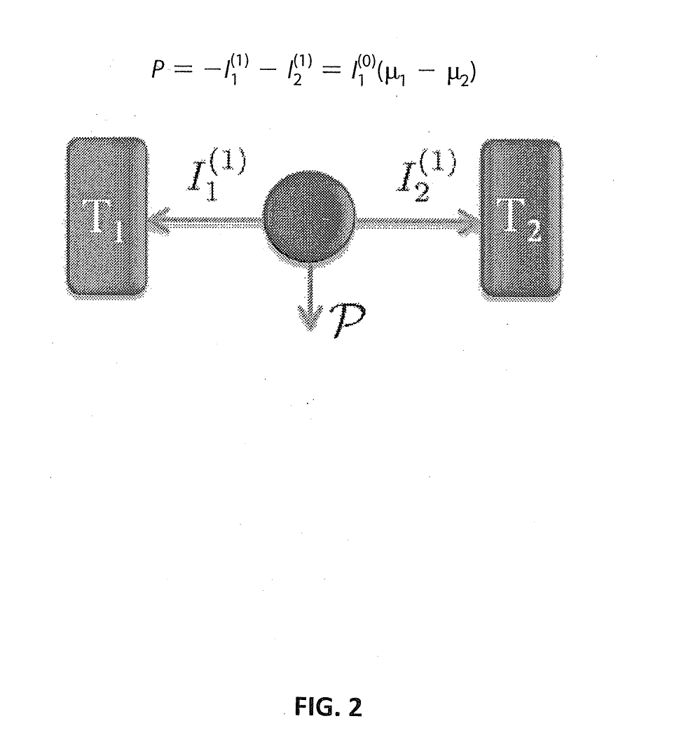

Thermodynamically, a system's response can be characterized by the efficiency η with which heat can be converted into usable power P and the amount of power that can be generated. An example of thermoelectric device is illustrated schematically in FIG. 2. In the figure, I_{α}⁽¹⁾ is the heat current flowing into lead α, T_{β} is the temperature and P is the power output. Applying the first law of thermodynamics to the device shown in FIG. 2 gives:

P=−I₁⁽¹⁾−I₂⁽¹⁾=I₁⁽⁰⁾(μ₁−μ₂), (8)

where we mention that the power can be equivalently phrased in terms of heat or electrical currents. The efficiency η can be defined as the ratio of power output to input heat current:

$$η = \frac{P}{\vert {I}_{1}^{( 1 )} \vert} = - \frac{{I}_{1}^{( 1 )} + {I}_{2}^{( 1 )}}{\vert {I}_{1}^{( 1 )} \vert} , \qquad ( 9 )$$

where we have assumed that T₁>T₂. With these expressions for the power and efficiency, the performance of a quantum device can be completely quantified, both near and far from equilibrium.

### a. Example Application to 1,3-Benzenedithiol-Au Junction

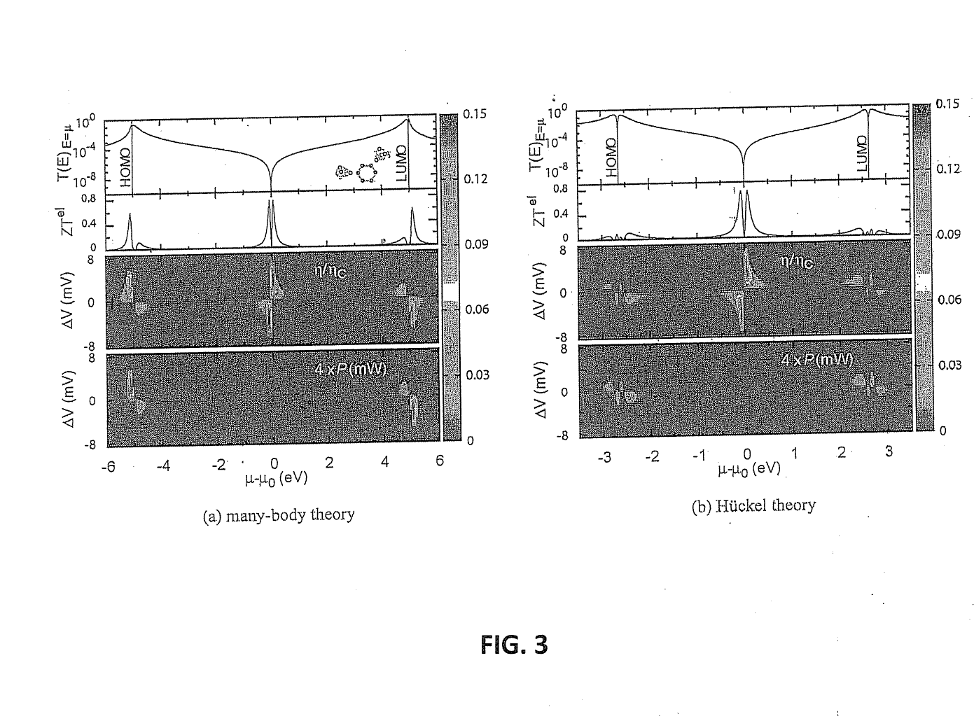

As a first example, we calculated the non-linear thermoelectric response of a meta-connected Au-benzene-Au SMJ using many-body and Hückel theory, shown in FIG. 3a and FIG. 3b, respectively. Although the transmission spectrum of this junction doesn't possess a supernode, it does possess a quadratic node within π-electron theory (Cardamone, D. M., Stafford, C. A., and Mazumdar, S., Controlling quantum transport through a single molecule. Nano Letters 2006, 6, 2422, hereinafter Cardamone2006), and allows us to ascertain the importance of interactions on the thermoelectric response of a SMJ.

Additional transport channels (e.g., from σ-orbitals) or incoherent scattering may lift the transmission node. The effect on the thermoelectric response is small provided these processes are weak. The effect of ρ-orbitals in SMJs whose π-orbitals exhibit an ordinary node was investigated in [Ke, S.-H., Yang, W., and Baranger, H. U., Quantum Interference Controlled Molecular Electronics. Nano Letters 2008, 8, 3257, hereinafter Ke2008]. Because the σ transmission is exponentially suppressed (Ke2008; Tao, N. J., Electron transport in molecular junctions, Nature Nanotechnology 2006, 1, 173-181, hereinafter Tao2006) as the length of the molecule increases, the effect of the σ-orbitals should be quantitatively insignificant in the biphenyl and larger molecules considered below.

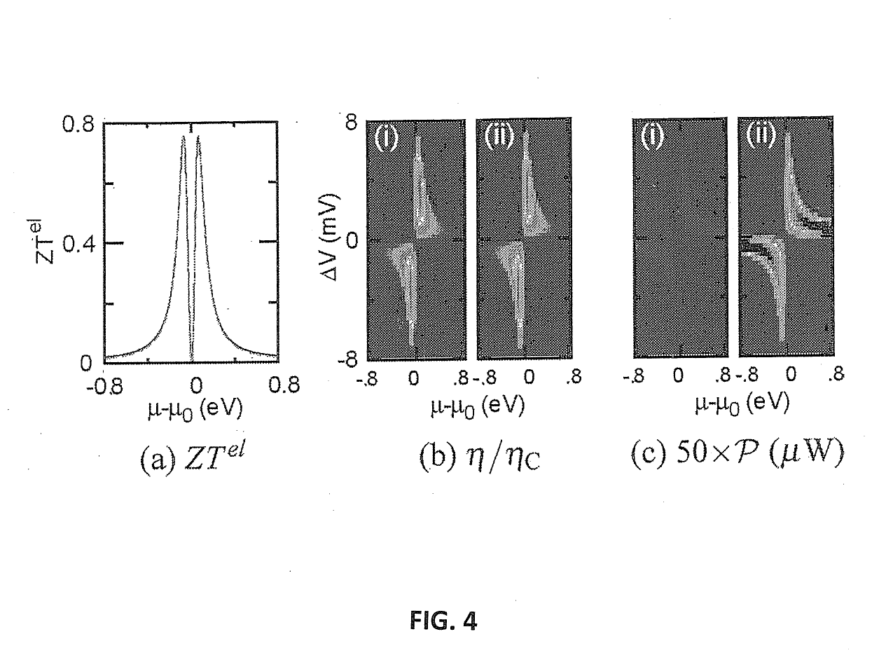

FIG. 3 illustrates thermoelectric characteristics of an example device based on a two-terminal 1,3-benzene Single-Molecule Junction, as determined from both many-body theory and Hückel theory. In the figure, the transmission probability T(E), figure-of-merit ZT^{el}, Carnot-normalized efficiency η/η_{C}, and electrical power output P of a two terminal 1,3-benzene SMJ, with lead temperatures T₁=300K and T₂=250K, are displayed as calculated using (a) many-body and (b) Hückel theory. The results highlight the discrepancies near resonances and the similarities near the node in the two theories. As a function of μ, η and ZT^{el} are in excellent qualitative agreement while P is only peaked near resonance, suggesting that ZT is incomplete as a device performance metric. The many-body calculations (a) give Pₚₑₐₖ=33 μW and ηₚₑₐₖ/η_{C}=11.5% near resonance. The Hückel theory calculations (b) give Pₚₑₐₖ=21 μW and ηₚₑₐₖ/η_{C}=2.7% near resonance. (The mid-gap region is discussed in FIG. 4.) Note that the peak ZT^{el}=0.75 is on par with currently available commercial thermoelectrics (Bell2008, 321; Snyder2008). The calculations were performed using Γ=0.63 eV.

In the top panel of FIG. 3 is a section of the transmission spectrum, showing the highest occupied molecular orbital (“HOMO”) and lowest unoccupied molecular orbital (“LUMO”) resonances and the quadratic node directly in between at μ=μ₀. Associated with this node is an enhancement in many linear-response metrics including ZT, which is shown in the second panel from the top. The bottom two portions of each figure show the calculated efficiency η and power P when a junction with T₁=300K and T₂=250K is further pushed out of equilibrium via the application of a bias voltage ΔV. In all calculations presented herein, the lead-molecule coupling is taken to be symmetric such that Γₙₘ^{α}=Γδₙₐδₘₐ, where n, m, and a are π-orbital labels and a is coupled to lead α. The efficiency is normalized with respect to the maximum allowed by the second law of thermodynamics, the Carnot efficiency η_{C}=ΔT/T₁, where ΔT=T₁−T₂.

The nonequilibrium thermodynamic response of a 1,3-benzene SMJ calculated using many-body theory is shown in FIG. 3a. The ZT and η spectra, shown in two middle panels of the same figure, exhibit peaks in the vicinity of both transmission nodes and resonances whereas the power P, shown in the bottom panel, is only peaked near transmission resonances. Around either the HOMU or LUMO resonance, the peak power Pₚₑₐₖ=33 μW and peak efficiency ηₚₑₐₖ/η_{C}=11.5% are realized when the junction operates out of equilibrium at a bias voltage ΔV=3 mV. With a chemical potential near the mid-gap node and ΔV=3.6 mV ηₚₑₐₖ/η_{C}=14.9%, larger than near resonance but with a much lower peak power Pₚₑₐₖ=0.088 nW.

In the vicinity of a resonance, there are both quantitative and qualitative differences in the linear and non-linear thermodynamic response predicted by the two theories. By neglecting interactions, the Hückel theory fails to accurately predict both the degeneracy and position of electronic resonances. It also incorrectly determines the peak values of ZT, η and P in the vicinity of a resonance. As can be seen near either (HOMO or LUMO) resonance in FIG. 2a, the Hückel theory predicts a Carnot-normalized peak efficiency of 2.7% which is nearly five times less than the 11.5% predicted by the many-body theory. The peak power near a resonance also varies considerably between the two theories, where the Hückel calculations give Pₚₑₐₖ=21 μW while many-body theory predicts Pₚₑₐₖ=33 μW. These results indicate that interactions should be taken into account in order to accurately predict the thermoelectric response of devices operating in the resonant-tunneling regime. It is interesting to note, however, that in both models the linear-response metric ZT qualitatively captures the features of the non-linear metric 17.

Of particular interest herein is the thermoelectric enhancement near nodes far away from any resonances. Although interactions ensure the invariance of transport quantities under a global voltage shift (i.e., gauge-invariance), near the particle-hole symmetric point the effect of interactions on the thermoelectric response should be small. In panels a-b of FIG. 4, a comparison of ZT and η using both many-body and Hückel theories is shown near μ₀ for a 1,3-benzene SMJ. Near this point, ZT and η are independent of theory employed. In contrast, the power, shown in panel c of the same figure, exhibits an order of magnitude difference between the two theories. This observation can be understood by noticing that the calculated HOMO-LUMO gap is ≈10 eV using many-body theory (panel c-i) whereas it is only ≈5.5 eV when interactions are neglected in the Hückel theory (panel c-ii). Since the power is peaked near transmission resonances, whose widths are fixed by the lead-molecule coupling Γ, the larger gap found using many-body theory gives a correspondingly lower predicted power.

More particularly, the calculations in FIG. 4 show ZT, η and P in the vicinity of the transmission node at μ=μ₀ of a meta-benzene SMJ using many-body (panel i) and Hückel (and panel ii) theories. In panels (a) and (b), ZT and η are found to be identical and independent of theory. In panel (c), P is strongly affected by interactions where, at peak efficiency (ηₚₑₐₖ/η_{C}μ14.91%), many body and Hückel calculations give Pₘₐₓ=0.088 nW and Pₘₐₓ=1.87 nW, respectively. The calculation parameters are the same as in FIG. 2.

While the Hückel theory does not accurately characterize the thermoelectric response of a junction in the resonant-tunneling regime, it is sufficient for predicting η and ZT in the vicinity of the transmission node. Moreover, Hückel theory is evidently valid for analysis of the larger molecules presented below.

The transmission node in a meta-benzene junction can be understood in terms of destructive interference of electron waves traversing the ring at the Fermi energy (Cardamone2006). According to Luttinger's theorem (Luttinger, J. M. Fermi Surface and Some Simple Equilibrium Properties of a System of Interacting Fermions. Phys. Rev. 1960, 119, 1153-1163, hereinafter Luttinger1960), the Fermi volume is unaffected by the inclusion of electron-electron interactions. Consequently, in an aromatic ring such as benzene the Fermi wavevector k_{F}=π/2d, where d is the inter-site distance, is conserved and is therefore sufficient to characterize quantum interference both with and without interactions near μ₀ since Δφ=k_{F}Δl, where Δφ is the relative phase between transport paths with length difference Δl.

### b. The Effect of Higher-Order Interferences

This last result indicates that the energy of resonant levels will generally depend strongly on whether or not interactions are included. Since k_{F} is protected, however, the transmission node across a single phenyl group is not so much a coincidence of energy levels as a wave phenomenon, meaning that interference in molecules composed of multiple aromatic rings in series can be understood in terms of the interference within each subunit rather than the energy spectrum of the entire molecule. We have found that such polycyclic molecules can exhibit higher-order supernodes, and that associated with a supernode is an order-dependent quantum enhancement of the junction's thermoelectric response.

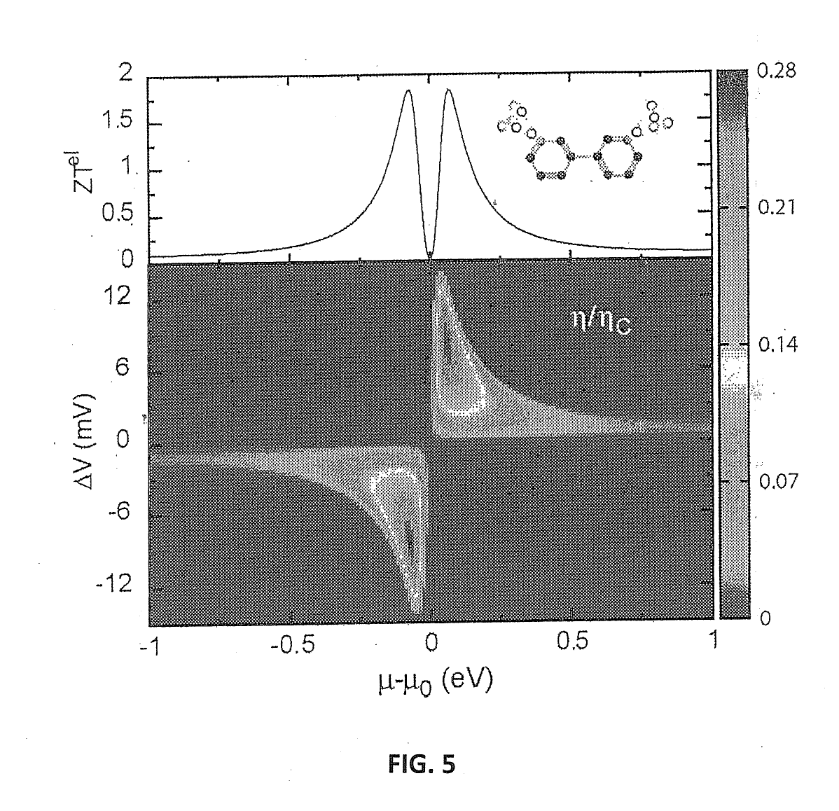

The 3,3′-biphenyl junction, drawn schematically in the top panel of FIG. 5, can be viewed as two meta-connected benzene rings in series. This junction geometry is similar to that studied by Mayor (Mayor, M., Weber, H. B., Reichert, J., Elbing, M., von Hänisch, C., Beckmann, D., and Fischer, M., Electric Current through a Molecular Rod—Relevance of the Position of the Anchor Groups. Angew. Chem. Int. Ed. 2003, 42, 5834-5838, hereinafter Mayor2003). In agreement with the prediction that a biphenyl junction should possess a quartic supernode, the linear and non-linear response shown in FIG. 5 exhibits peak values of efficiency (η/η_{C}=26.86%) and ZT^{el} (1.84) that are over twice those of benzene. With ZT≈2, the biphenyl junction exhibits sufficient thermoelectric performance to be attractive for many commercial solid-state heating and cooling applications. We further discovered that this is only the first in an entire class of supernode-possessing molecules which exhibit even larger values of η and ZT.

In larger molecules composed of n meta-connected phenyl groups in series, we expect that the transmission nodes should combine and give rise to a 2n^{th} order supernode. This is demonstrated in FIG. 5, which shows a closeup of ZT and η near the quartic supernode of a 3,3′-biphenyl SMJ with ZTₚₑₐₖ=1.84 and ηₚₑₐₖ/η_{C}=26.86% at a predicted power of 0.75 pW. The junction geometry is shown schematically in the inset of the upper panel. Calculations were performed using Hückel theory with T₁=300K, T₂=250K and Γ=0.5 eV.

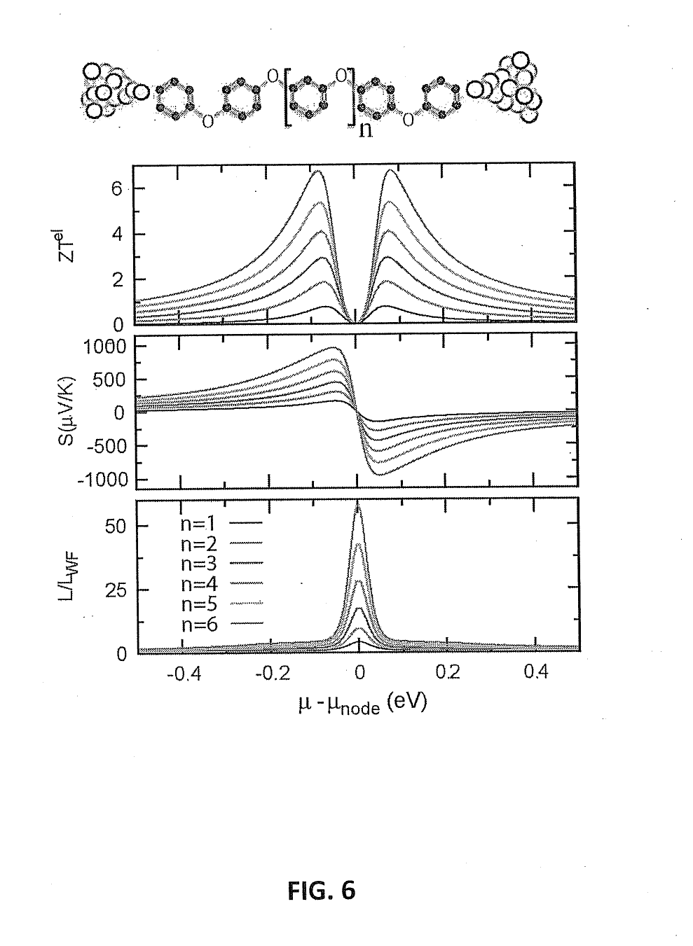

More particularly, a polyphenyl ether (“PPE”) is shown schematically at the top of FIG. 6 consisting, by way of example, of n phenyl rings connected in series with ether linkages. Based on the discussion above, a PPE-based junction can be predicted to exhibit a 2n^{th} order supernode. The figure-of-merit ZT, thermopower S and Lorenz number L=κ/GT for PPE junctions are shown in the top, middle and bottom panels of FIG. 6, respectively, where the Lorenz number is normalized with respect to the Wiedemann—Franz (“WF”) value L_{WF}=(π²/3)(k_{B}/e)².

FIG. 6 illustrates a supernode enhancement of ZT, thermopower S and Lorenz number L for polyphenyl ether (PPE) SMJs with n repeated phenyl groups, again shown schematically above the top panel. As a function of n, ZTₚₑₐₖ scales super-linearly exhibiting a peak value of 6.86 for n=6. The thermopower and Lorenz number are also enhanced with Sₚₑₐₖ=957 μV/K and Lₚₑₐₖ=55.33 L_{WF} at the same value of n. Calculations were performed using Hückel theory at room temperature (T=300K) with Γ=0.5 eV. Inter-phenyl electronic hopping was set an order of magnitude below the intra-phenyl value of 2.64 eV.

The bottom panel of FIG. 6 shows an increasing peak Lorenz number Lₚₑₐₖ with increasing n. In linear-response, L and S can be expressed in terms of 6 as:

$${L \vert}_{el} = \frac{1}{{( eT )}^{2}} ( \frac{{L}^{( 2 )}}{{L}^{( 0 )}} - {[ \frac{{L}^{( 1 )}}{{L}^{( 0 )}} ]}^{2} ) , \\ and S = - \frac{1}{eT} \frac{{L}^{( 1 )}}{{L}^{( 0 )}} , \qquad ( 10 )$$

where e is the magnitude of the electron's charge and T is the temperature. Using Eq 10 and Eq 6 with the transmission function of Eq 1 we determined that:

$${\frac{{L}_{ma x}}{{L}_{WF}} \vert}_{el} = ( \frac{3}{{π}^{2}} ) \frac{{[ {\partial }_{b}^{2 n + 2} b π csc ( b π ) ] \vert}_{b = 0}}{{[ {\partial }_{b}^{2 n} b π csc ( b π ) ] \vert}_{b = 0}} . \qquad ( 11 )$$

Setting n=6 in 11 gives Lₘₐₓ=55.33 L_{WF}, corresponding exactly to the result of the full calculation shown in the bottom panel of FIG. 6. Similar agreement has been found for the other values of n, confirming the presence of 2n^{th} order supernodes in these junctions.

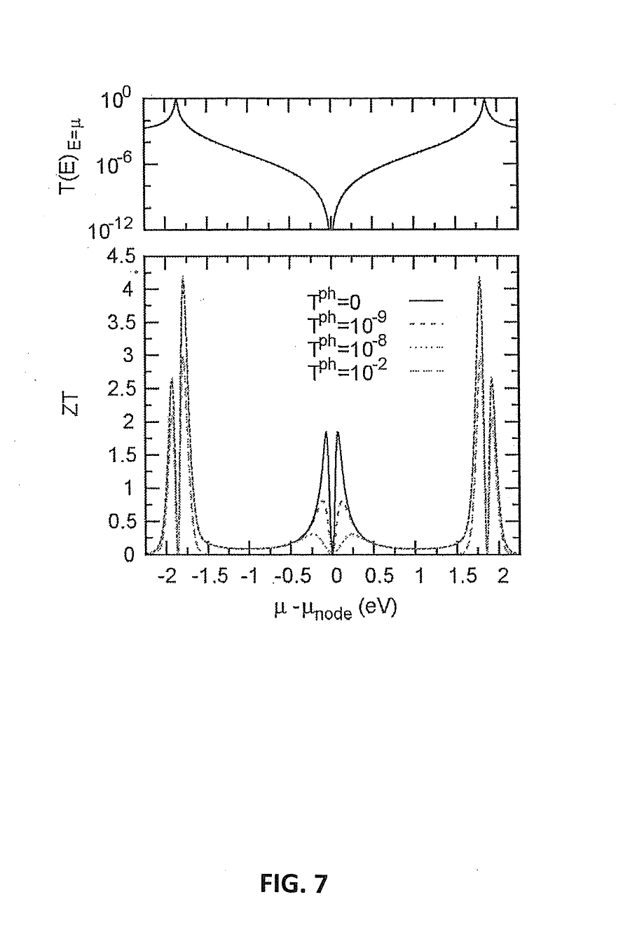

The above discussion considered purely electronic transport. According to Eq 4, phonon heat transport may reduce ZT significantly (Liu2009), although it should be emphasized that the thermopower of the junction is unaffected provided the electron-phonon coupling is negligible. FIG. 7 shows the effect of phonon heat transport on ZT of a 3,3′-biphenyl junction for several values of the phonon transmission probability T^{ph}. The transmission probability T(E) and ZT are shown for a 3,3′-biphenyl SMJ with several different phonon transmission values. Although phonon transport strongly reduces ZT near the supernode, the enhancement of ZT near the transmission peaks is fairly insensitive to moderate values of T^{ph}. Recall that κ^{ph}=κ₀T^{ph}.

In the vicinity of the quartic transmission node, ZT is significantly reduced even for small values of T^{ph}. However, the large peaks of ZT found near the transmission resonances are largely insensitive to phonon heat transport due to the smaller ratio of κ^{ph}/κ^{el}. Accordingly, the inventors have determined that practical supernode-based devices will need careful engineering of phonon transport. For example, the inclusion of a vacuum tunneling gap in series with the junction would effectively block phonon transport.

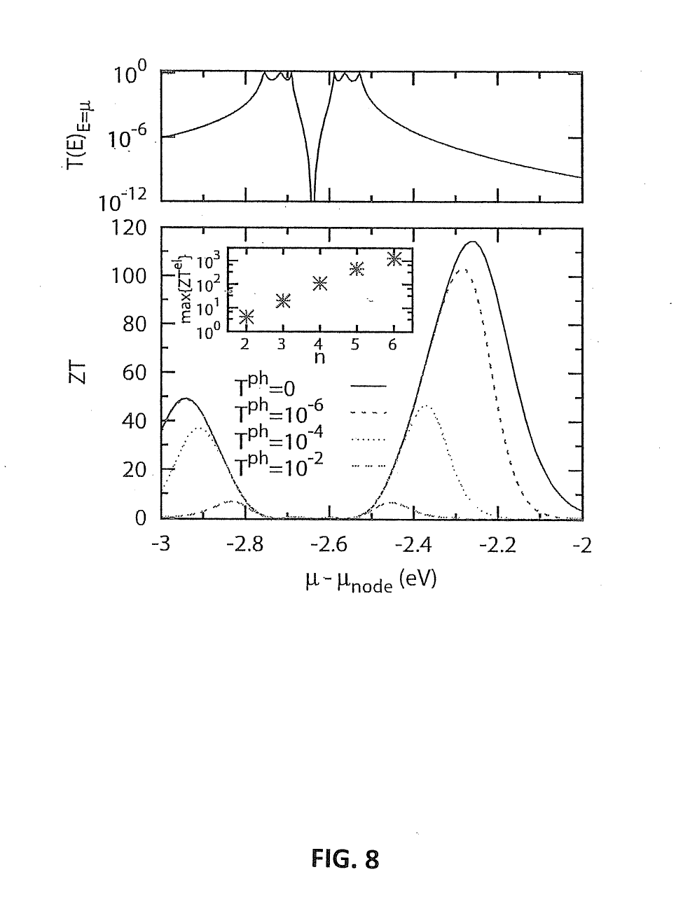

Higher-order quantum interference effects can arise from both destructive and constructive interference. As evidenced by FIG. 7, thermoelectric devices based on constructive interference are far less sensitive to phonon effects. FIG. 8 shows the transmission spectrum and ZT^{el} near the HOMO resonance of a tetraphenyl ether SMJ. In particular, the transmission and ZT in the vicinity of a transmission peak for a tetraphenyl ether (n=4) molecule shows that ZT^{el} is enhanced in the vicinity of a higher-order peak.

The transmission resonance exhibits fine structure due to electronic standing waves along the molecular chain (Kassubek, F., Stafford, C. A., and Grabert, H., Force, charge, and conductance of an ideal metallic nanowire. Phys. Rev. B 1999, 59, 7560-7574, hereinafter Kassubek1999). The interplay of the many closely spaced resonances gives rise to a dramatic enhancement of the thermopower in a regime of large electrical conductance, and hence a very large ZT^{el}: 10². The inset of FIG. 8 shows the exponential scaling of the peak ZT^{el} near the HOMO resonance of a polyphenyl ether SMJ as a function of the phenyl group number n. The predicted giant enhancement of ZT^{el} occurs over a broad energy range, in contrast to that expected from a narrow transmission resonance (Finch2009).

### 2. Example Embodiments of Enhanced Thermoelectric Devices

The higher-order quantum interferences in the transmission spectrum of a nanoscale junction give rise to an order-dependent quantum-enhancement of the linear and non-linear thermoelectric response. The full nonequilibrium spectrum of thermodynamic efficiency qualitatively resembles the figure-of-merit ZT spectrum, suggesting that ZT encapsulates the salient physics related to efficiency even at the nanoscale. Beyond efficiency, another important quantity is the usable power produced by a device, variations of which are not likely to be reliably characterized by ZT alone at the nanoscale.

Thermoelectric devices based on individual SMJs or other quantum conductors exhibiting coherent electronic transport are ideally suited for local cooling in integrated nanoscale circuit architectures. Supernode-based devices have a low transmission probability and thus a large electrical impedance capable of withstanding voltage surges, while devices based on higher-order constructive interference are more robust with respect to phonon heat transport. Embodiments of such high-power macroscopic devices could be constructed by growing layers of densely packed molecules. For example, a self-assembled monolayer with a surface density (Zangmeister, C. D., Robey, S. W., van Zee, R. D., Yao, Y., Tour, J. M. Fermi Level Alignment and Electronic Levels in Molecular Wire Self-Assembled Monolayers on Au, The Journal of Physical Chemistry B 2004, 108, 16187-16193, hererinafter Zangmeister2004) of 4×10¹⁵ molecules/cm² would give 352 kW/cm² at peak efficiency for a meta-benzene film. Furthermore, the efficiency of PPE-based devices increases with ring number and may be limited only by the electronic coherence length and phonon heat transport.

The theoretical foundations discussed above provide a basis and serve as a guide for embodiments of practical, nanoscale thermoelectric devices that take advantage of the enhanced effects. Example embodiments of such devices are described below. In view of the applicability of the physical principles to a wide range of materials and configurations, it will be appreciated that the example embodiments discussed below should not be viewed as limiting with respect to employing high-order quantum interference in thermoeletric devices.

a. Generic Device Configuration for a Nanoscale Thermoelectric Device

In accordance with the physical principles discussed above, an example embodiment of a thermoelectric device will include a first electrode, a second electrode, and an electrical transmission medium electrically connected to the first and second electrodes. In particular, the electrical transmission medium will be a quantum conductor that exhibits at least one transmission node or transmission resonance due to quantum interference. Transmission nodes or transmission resonances arising from coherent electronic transport imbue the quantum characteristics described above into the electrical transmission medium, so that a junction formed by the connection of the first and second electrodes via the electrical transmission medium will exhibit enhanced thermoelectric response.

In further accordance with the example embodiment, the thermoelectric device may be configured to be operable as a thermoelectric power generator. More particularly, in response to a temperature difference between the first and second electrodes, the device will develop a voltage difference between the two electrodes. This configuration thus implements the Seebeck effect on the scale of junction. In practice, the first electrode will be configured to be in thermal contact with a heat source, and the second electrode will be configured to be in thermal contact with an ambient temperature reservoir. By usual convention a heat source is taken to be at a higher temperature than the ambient temperature reservoir (referred to herein simply as an “ambient”). Accordingly, the first electrode will be at a higher temperature than the second electrode.

In still further accordance with the example embodiment, the thermoelectric device may be configured to be operable as a Peltier cooler, having a low-temperature side and a high-temperature side. In this configuration, the thermoelectric device will transfer heat from the low-temperature side to the high temperature side in response to a voltage applied between the first and second electrodes, thereby cooling the low-temperature side. The Peltier cooler may be considered a power generator running in reverse, consuming electric energy to effect transfer of heat across the junction between the two electrodes.

The quantum conductor could be an organic molecule bonded to the first and second electrodes, thereby forming a SMJ between the two electrodes. More particularly, the organic molecule could include a plurality of meta-connected benzene rings. Such a molecule would exhibit enhanced thermoelectric response, in accordance with the physical principles discussed above. In practice, the electrical transmission medium could include a plurality of organic molecules bonded to the first and second electrodes. By way of example, the plurality of organic molecules could be arranged in a self-assembled monolayer (“SAM”).

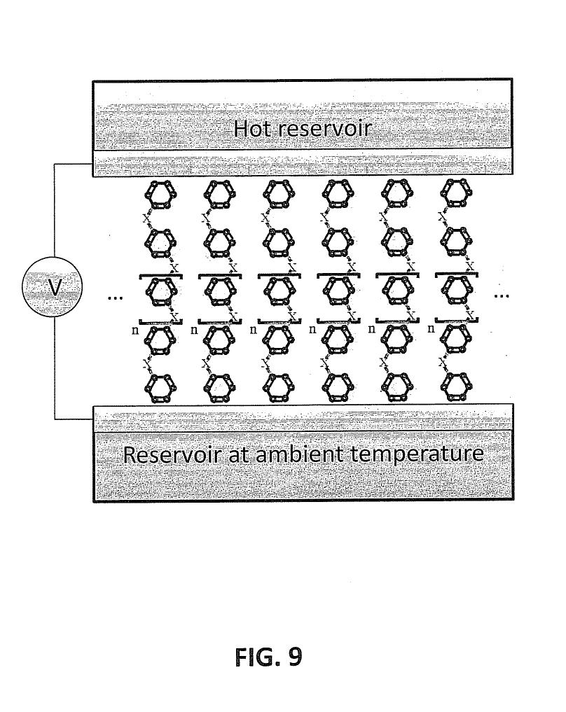

FIG. 9 illustrates such an arrangement. As shown, the top end of the device includes a first electrode in thermal contact with a hot reservoir, and the bottom end of the device includes a second electrode in thermal contact with a reservoir at ambient temperature (where “top” and “bottom” are referenced with respect to the orientation of the figure, and do not necessarily imply any intrinsic properties of the device). Each of a plurality of single molecules is bonded to the first and second electrodes, such that a plurality of parallel connections is formed between the electrodes. In accordance with the example embodiment, each single molecule includes one or more meta-connected benzene rings, each molecule thereby possessing the physical attributes that give rise to supernodes or resonances that yield enhanced thermoelectric response in the junction between the electrodes. The ellipses in the figure indicate that there could be more molecules than those in the illustration.

When the device is operated as a power generator, a voltage is developed between the electrodes in response to a flow of heat from the hot reservoir to the ambient reservoir. Alternatively, when the device is operated as a Peltier cooler, heat is transferred from a cold reservoir to the hot reservoir in response to a voltage applied between the two electrodes. In either case, the enhanced thermoelectric response arising from the quantum interference effects of the SMJs advantageously results in enhanced efficiency and performance of the thermoelectric device.

b. Power Generator and Peltier Cooler

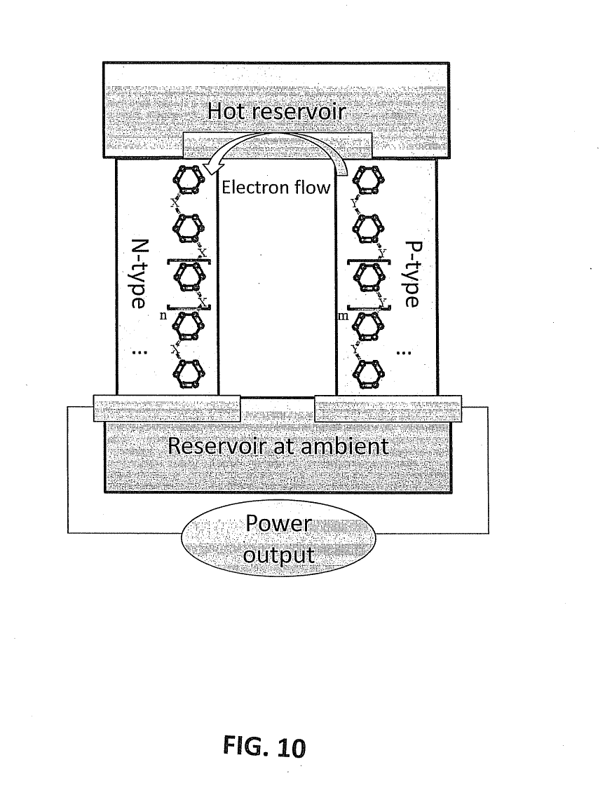

An alternative embodiment of a power generator is depicted in FIG. 10, in which the thermoelectric device now includes a third electrode electrically connected to the electrical transmission medium. In accordance with the alternative embodiment, the electrical transmission medium includes a first quantum conductor between the first and second electrodes and a second quantum conductor between the second and third electrodes. As shown, the first and third electrodes are on a first side of the device, and the second electrode is on a second side of the device. For purposes of illustration in FIG. 10, the first side is configured at the bottom of the device and the second side is configured at the top (where “top” and “bottom” are referenced with respect to the orientation of the figure, and do not necessarily imply any intrinsic properties of the device). In this configuration, the first and second quantum conductors are connected in series between the first and third conductors, via the second conductor.

In further accordance with the alternative embodiment, the first quantum conductor includes an N-type organic molecule and the second quantum conductor includes a P-type organic molecule. More particularly, the N-type organic molecule is bonded to the first and second electrodes and the P-type organic molecule is bonded to the second and third electrodes, as depicted in FIG. 10. In practice, the electrical transmission medium of the alternative embodiment will include a plurality of N-type organic molecules bonded to the first and second electrodes and a plurality of P-type organic molecules bonded to the second and third electrodes.

As with the embodiment described in connection with FIG. 9, each organic molecule will include a meta-connected benzene ring, such that the plurality of N-type organic molecules forms a plurality of SMJs connected in parallel between the first and second electrodes, and the plurality of P-type organic molecules forms a plurality of SMJs connected in parallel between the second and third electrodes. In this configuration, the N-type organic molecules individually comprise electron donor substituents on a backbone of meta-connected benzene rings and the P-type organic molecules individually comprise electron acceptor substituents on a backbone of meta-connected benzene rings. Although only one of each type of molecule is shown in FIG. 10, the ellipses in the figure indicate that each may represent a respective plurality. Again, the N-type organic molecules and P-type organic molecules may be arranged in SAMs.

The alternative embodiment can be operated as a thermoelectric power generator by providing a temperature difference between the first and second sides of the device. More specifically, a voltage difference between the first and third electrodes will be developed in respond to a temperature difference between the first and second sides. In accordance with the alternative embodiment, the second side will be in thermal contact with a heat source and the first side will be in thermal contact with an ambient-temperature reservoir, such that the first and third electrodes are at a lower temperature than the second electrode. Then, a voltage difference between the first and third electrodes will be generated in response to a flow of heat across the junction between the first and second sides. The enhanced thermoelectric response arising from the quantum interference effects of the N-type and P-type SMJs advantageously results in enhanced efficiency and performance of the thermoelectric power generator.

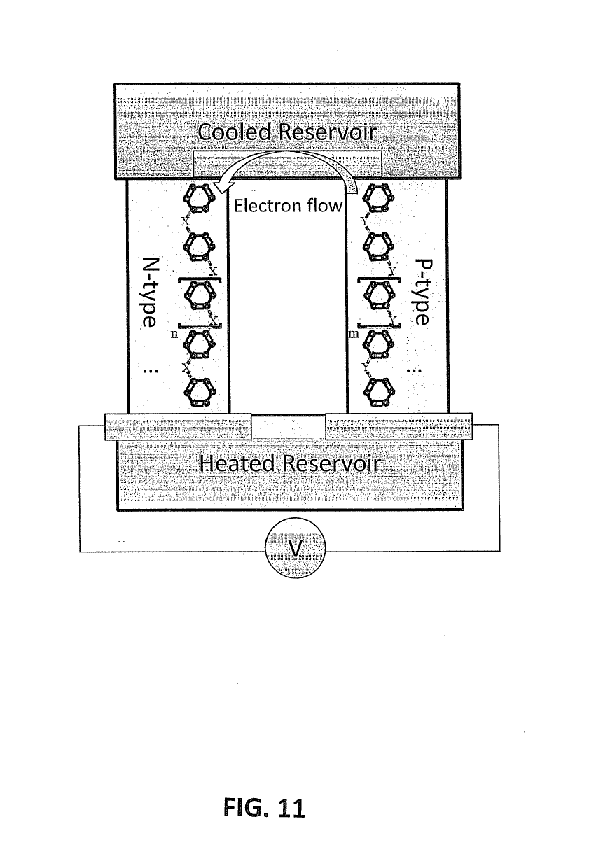

The alternative embodiment of a thermoelectric power generator can be run in reverse as a Peltier cooler. Such an arrangement is shown in FIG. 11, where the indicated voltage applied between the first and third electrodes now causes heat to flow from the low-temperature second side to the high-temperature first side. By fabricating the first and second quantum conductors from molecules exhibiting high-order quantum interference supernodes or resonances, the efficiency and performance of the Peltier cooler is again enhanced.

c. Molecular structures

Compounds that may be used in the devices and assemblies described herein include compounds of formula (I),

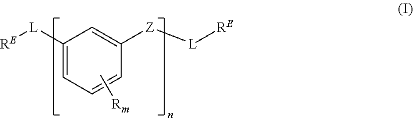

wherein

n is 1-100;

Z is a bond, —O—, —S—, —N(R^{Z})—, —C(O)—, —S(O)—, —S(O)₂—, —C(R^{Z})₂—, —C(R^{Z})═C(R^{Z})—, —C≡C—, wherein each R^{Z} is independently hydrogen or C₁-C₆ alkyl;

each m is independently 0, 1, 2, 3, or 4;

each R is independently an electron-donating group, an electron-withdrawing group, or a group electrically similar to hydrogen;

each L is independently a bond or a divalent linking group;

each R^{E} is independently a functional group capable of bonding to or associating with a metal surface.

In certain embodiments, each R is independently an electron-donating group or an electron-withdrawing group.

In certain embodiments, the compounds of formula (I) are considered “N-type” as described above when at least one R group is an electron-donating substituent as is familiar to those skilled in the art. In certain embodiments, the compounds of formula (I) are “N-type” when each R group is an electron-donating substituent. An “electron-donating group” refers to a functional group that donates electrons to a neighboring atom more than a hydrogen atom would if it occupied the same position in a molecule. Examples of electron-donating substituents include, but are not limited to C₁-C₆ alkyl, —OR¹, —N(R¹)₂, or —SR¹.

In other embodiments, the compounds of formula (I) are considered “P-type” as described above when at least one R group is an electron-withdrawing substituent as is familiar to those skilled in the art. In other embodiments, the compounds of formula (I) are considered “P-type” when each R group is an electron-withdrawing substituent. An “electron-withdrawing group” refers to a functional group that draws electrons to itself more than a hydrogen atom would if it occupied the same position in a molecule. Examples of electron-withdrawing substituents include, but are not limited to halogen, cyano, nitro, trifluoromethyl, —C(O)OR¹, —C(O)R¹, —C(O)N(R¹)₂, —S(O)₂R¹, —S(O)₂N(R¹)₂, —S(O)₂OR¹.

In other embodiments, the compounds of formula (I) can be engineered to include substituents that are electrically similar but whose masses are different to modify the vibrational spectrum to limit the phonon thermal conductance through the junctions described herein (i.e., “phonon engineering”). For example, substitutions to one or several phenyl groups along the backbone can be made so that the masses of the various phenyl groups (taken to include the hydrogens groups and R groups) vary in an irregular fashion. The vibrational modes of the compounds of formula (I) then can tend to be localized, and the phonon thermal conductance of the compounds in the junctions can be suppressed. These substituents (i.e., R groups) can be selected to simultaneously alter the electrical properties of the molecule (e.g. R groups selected as n-type or p-type “doping”) and to suppress phonon thermal transport (e.g., electrically similar, but differing mass).

In one particular example, substituents for one or more of the hydrogens in the phenyl groups in any of the compounds described herein can be replaced with a substituent having an electronegativity similar to hydrogen (i.e., it is “electrically similar” to hydrogen).

The term “electrically similar” as used herein mean that the referenced entities have a Pauling electronegativity (χ) of about +/−0.10 of one another; such can be used in an aggregate sense, for example, the electronegativities of a set of substituents can be summed and compared to a second set of substituents to determine if the sets, each taken as a whole, are electrically similar to one another (i.e., the sums are about +/−0.10 of one another). For example, an entity that is “electrically similar” to hydrogen has χ≈2.20+/−0.10. In a particular example, replacing hydrogen for a methyl group (χ×2.30) leads to a small change in the thermoelectric response of a benzenedithiol junction indicating that a methyl group is electrically similar to hydrogen. Since the mass of the methyl group is about 15 times greater than that of hydrogen, such a substituent would significantly alter the vibrational spectrum of a molecule with a backbone of phenyl groups. Such substitutions may also be made in the linker groups (“L” as defined herein).

Where phenyl groups in the compounds herein have m=1-4, for example, to achieve n-type or p-type doping, then the overall electron-donating (or accepting) character of the substituents on each phenyl group can be electrically similar, but having the total masses of the substituents (i.e., the sum of the masses of any hydrogens and R groups on each phenyl) varying from one phenyl group to the next. The transmission of (quantum) sound waves can be reduced along the backbone of the molecule in that the mass per unit length should not be periodic, but should be random, or have one or several groups with mass differing greatly from the others.

A “linking group” as used herein means any divalent organic moiety capable of connecting an end group, (R^{E}) as defined herein, to the core of the parent compound. Examples of linking groups include, but are not limited to, polymers, peptides, oligomers, dendrimers, where the end group and the core of the parent compound are each bonded to an available position within the linking group. Other examples of linking groups include groups of the formula, —(C₀-C₁₀ alkyl-J)₀₋₂—, wherein each J is independently a bond, aryl, heteroaryl, C₃-C₈ cycloalkyl, or heterocyclyl; and no more than one methylene in each alkyl group is optionally and independently replaced by —O—, —S—, —N(R⁰)—, —C(H)═C(H)—, —C≡C—, —C(O)—, —S(O)—, —S(O)₂—, —P(O)(OH)—, —OP(O)(OH)—, —P(O)(OH)O—, —N(R⁰)P(O)(OH)—, —P(O)(OH)N(R⁰)—, —OP(O)(OH)O—, —OP(O)(OH)N(R⁰)—, —N(R⁰)P(O)(OH)O—, —N(R⁰)P(O)(OH)N(R⁰)—, —C(O)O—, —C(O)N(R⁰)—, —OC(O)—, —N(R⁰)C(O)—, —S(O)O—, —OS(O)—, —S(O)N(R⁰)—, —N(R⁰)S(O)—, —S(O)₂O—, —OS(O)₂—, —S(O)₂N(R⁰)—, —N(R⁰)S(O)₂—, OC(O)O—, —OC(O)N(R⁰)—, —N(R⁰)C(O)O—, —N(R⁰)C(O)N(R⁰)—, —OS(O)O—, —OS(O)N(R⁰)—, —N(R⁰)S(O)O—, —N(R⁰)S(O)N(R⁰)—, —OS(O)₂O—, —OS(O)₂N(R⁰)—, —N(R⁰)S(O)₂O—, or —N(R⁰)S(O)₂N(R⁰)—, wherein each R⁰ is independently hydrogen or C₁-C₆

A “functional group capable of bonding to or associating with a metal surface” as used herein refers to chemical entities that include at least one chemical group capable of reacting with or coordinating to a metal layer surface. Examples of suitable functional groups for bonding or coordinating to metals include, but are not limited to, —NH₂, —COOH, —OH, —SH, and chemical compounds containing the same.

In an embodiment of any of the preceding embodiments of formula (I), Z is bond, —O—, —S—, or —N(R^{Z})—. In certain embodiments, Z is bond or —O—. In certain other embodiments, Z is bond. In certain other embodiments, Z is —O—.

In an embodiment of any of the preceding embodiments of formula (I), each R^{E} is independently halogen, —OH, —COOH, —CN, —NH₂, —N±N⁺(Y⁻), —SH, —S₂O₃⁻Na⁺, —SAc,

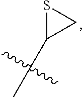

### —SR¹, —SSR¹,

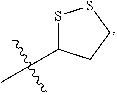

—C(S)SH, —SeH, —SeSe R¹, —PR¹₂, —P(O)R¹₂, —P(O)(O—)₂²⁻, —P(O)(OH)₂, —PO₄²⁻, —N≡C, —C(H)═CH₂, —C≡CH, —SiX₃, —Si(OR¹)₃, wherein X is hydrogen or halogen; and Y is a halide, perchlorate, tetrafluoroborate, or hexafluorophosphate and wherein each R¹ is independently hydrogen, C₁-C₆ alkyl, or C₁-C₆ haloalkyl;

In an embodiment of any of the preceding embodiments of formula (I), each R^{E} is independently halogen, —SH, —COOH, —P(O)(OH)₂, —SiX₃, —Si(OR¹)₃, —C≡N, or —N≡N⁺(Y⁻), wherein X is halogen; and Y is a halide, perchlorate, tetrafluoroborate, or hexafluorophosphate. In certain embodiments, each R^{E} is independently halogen or —N≡N⁺(Y⁻). In certain other embodiments, each R^{E} is independently —SiX₃ or —Si(OR¹)₃, wherein X is halogen. In certain other embodiments, each R^{E} is independently —SH, —COOH, or —P(O)(OH)₂. In an embodiment of any of the preceding embodiments of formula (I), each R^{E} is independently —SH, —S₂O₃⁻Na⁺, —SAc,

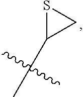

### —SR¹, —SSR¹,

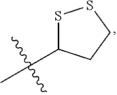

or —C(S)SH. In certain other embodiments, each R^{E} is —SH.

In an embodiment of any of the preceding embodiments of formula (I), each L is independently of the formula, —(C₀-C₁₀ alkyl-J)₀₋₂—, wherein each J is independently a bond, aryl, heteroaryl, C₃-C₈ cycloalkyl, or heterocyclyl; and no more than one methylene in each alkyl group is optionally and independently replaced by —O—, —S—, —N(R⁰)—, —C(H)═C(H)—, —C≡C—, —C(O)—, —S(O)—, —S(O)₂—, —P(O)(OH)—, —OP(O)(OH)—, —P(O)(OH)O—, —N(R⁰)P(O)(OH)—, —P(O)(OH)N(R⁰)—, —OP(O)(OH)O—, —OP(O)(OH)N(R⁰)—, —N(R⁰)P(O)(OH)O—, —N(R⁰)P(O)(OH)N(R⁰)—, —C(O)O—, —C(O)N(R⁰)—, —OC(O)—, —N(R⁰)C(O)—, —S(O)O—, —OS(O)—, —S(O)N(R⁰)—, —N(R⁰⁰)S(O)—, —S(O)₂O—, —OS(O)₂—, —S(O)₂N(R⁰)—, —N(R⁰)S(O)₂—, OC(O)O—, —OC(O)N(R⁰)—, —N(R⁰)C(O)O—, —N(R⁰)C(O)N(R⁰)—, —OS(O)O—, —OS(O)N(R⁰)—, —N(R⁰)S(O)O—, —N(R⁰)S(O)N(R⁰)—, —OS(O)₂O—, —OS(O)₂N(R⁰)—, —N(R⁰)S(O)₂O—, or —N(R⁰)S(O)₂N(R⁰)—, wherein each R⁰ is independently hydrogen or C₁-C₆ alkyl.

In certain embodiments, each L is independently —C₀-C₁₀ alkyl-J-C₀-C₁₀ alkyl-J-, wherein each J is independently a bond, aryl, heteroaryl, C₃-C₈ cycloalkyl, or heterocyclyl; and no more than one methylene in each alkyl group is optionally and independently replaced by —O—, —S—, —N(R⁰)—, —C(H)═C(H)—, —C≡C—, —C(O)—, —S(O)₂—, —C(O)O—, —C(O)N(R⁰)—, —OC(O)—, —N(R⁰)C(O)—, —S(O)₂N(R⁰)—, —N(R⁰)S(O)₂—, OC(O)O—, —OC(O)N(R⁰)—, —N(R⁰)C(O)O—, —N(R⁰)C(O)N(R⁰)—, wherein each R⁰ is independently hydrogen or C₁-C₆ alkyl.

In certain other embodiments, each L is independently —C₀-C₁₀ alkyl-J-C₀-C₁₀ alkyl-J-, wherein each J is independently a bond or aryl and no more than one methylene in each alkyl group is optionally and independently replaced by —O—, —S—, —N(R⁰)—, —C(H)═C(H)—, —C≡C—, —C(O)—, —S(O)₂—, —C(O)O—, —C(O)N(R⁰)—, —OC(O)—, —N(R⁰)C(O)—, —S(O)₂N(R⁰)—, —N(R⁰)S(O)₂—, OC(O)O—, —OC(O)N(R⁰)—, —N(R⁰)C(O)O—, —N(R⁰)C(O)N(R⁰)—, wherein each R⁰ is independently hydrogen or C₁-C₆ alkyl.

In yet other embodiments, each L is independently of the formula,

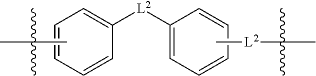

wherein each L² is independently a bond, —CH₂—, —O—, —S—, —N(R⁰)—, —C(H)═C(H)—, —C≡C—, —C(O)—, —S(O)₂—, —C(O)O—, —C(O)N(R⁰)—, —OC(O)—, —N(R⁰)C(O)—, —S(O)₂N(R⁰)—, —N(R⁰)S(O)₂—, OC(O)O—, —OC(O)N(R⁰)—, —N(R⁰)C(O)O—, or —N(R⁰)C(O)N(R⁰)—.

In yet other embodiments, each L is independently of the formula,

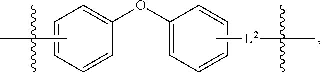

wherein L² is a bond or —O—.

In an embodiment of any of the preceding embodiments of formula (I), each R is independently halogen, cyano, nitro, C₁-C₆ alkyl, C₁-C₆ haloalkyl, —N(R¹)₂, —SR¹, —C(O)OR¹, —C(O)R^{D}, —C(O)N(R¹)₂, —S(O)R^{B}, —S(O)₂R¹, —S(O)N(R¹)₂, —S(O)₂N(R¹)₂, —S(O)OR¹, —S(O)₂OR¹, —OC(O)R¹, —OC(O)OR¹, —OC(O)N(R¹)₂, N(R¹)C(O) R¹, —N(R¹)C(O)OR¹, —N(R¹)C(O)N(R¹)₂, C₃-C₈ cycloalkyl, heterocyclyl, aryl, or heteroaryl, wherein each R¹ is independently hydrogen, C₁-C₆ alkyl, or C₁-C₆ haloalkyl.

In an embodiment of any of the preceding embodiments of formula (I), each R is independently halogen, cyano, nitro, C₁-C₄ alkyl, C₁-C₄ haloalkyl, —N(R¹)₂, —SR¹, —C(O)OR¹, —C(O)R¹, —C(O)N(R¹)₂, —S(O)₂R¹, —S(O)₂N(R¹)₂, or —S(O)₂OR¹, wherein each R¹ is independently hydrogen, C₁-C₆ alkyl, or C₁-C₆ haloalkyl.

In an embodiment of any of the preceding embodiments of formula (I), wherein each m is independently 0, 1, or 2; and each R is independently halogen, cyano, nitro, trifluoromethyl, C(O)OR¹, —C(O)R¹, —C(O)N(R¹)₂, —S(O)₂R¹, —S(O)₂N(R¹)₂, —S(O)₂OR¹.

In an embodiment of any of the preceding embodiments of formula (I), each m is independently 0, 1, or 2; and each R is independently C₁-C₆ alkyl, —OR¹, —N(R¹)₂, or —SR¹.

In an embodiment of any of the preceding embodiments of formula (I), at least one R group, when present, is electrically similar to hydrogen. In an embodiment of any of the preceding embodiments of formula (I), at least one R group, when present, is C₁₋₆ alkyl (e.g., methyl or tert-butyl).

In an embodiment of any of the preceding embodiments of formula (I), at least one R group, when present is electrically similar to hydrogen, and the remaining R groups are each independently halogen, cyano, nitro, C₁-C₆ alkyl, C₁-C₆ haloalkyl, —N(R¹)₂, —SR¹, C(O)OR¹, —C(O)R^{D}, —C(O)N(R¹)₂, —S(O)R^{B}, —S(O)₂R¹, —S(O)N(R¹)₂, —S(O)₂N(R¹)₂, —S(O)OR¹, —S(O)₂OR¹, —OC(O)R¹, —OC(O)OR¹, —OC(O)N(R¹)₂, —N(R¹)C(O)R¹, —N(R¹)C(O)OR¹, —N(R¹)C(O)N(R¹)₂, C₃-C₈ cycloalkyl, heterocyclyl, aryl, or heteroaryl, wherein each R¹ is independently hydrogen, C₁-C₆ alkyl, or C₁-C₆ haloalkyl.

In an embodiment of any of the preceding embodiments of formula (I), at least one R group, when present is C₁₋₆ alkyl (e.g., methyl or tert-butyl), and the remaining R groups are each independently halogen, cyano, nitro, C₁-C₆ alkyl, C₁-C₆ haloalkyl, —OR¹, —N(R¹)₂, —SR¹, —C(O)OR¹, —C(O)R¹, —C(O)N(R¹)₂, —S(O)R^{B}, —S(O)₂R¹, —S(O)N(R¹)₂, —S(O)₂N(R¹)₂, —S(O)OR¹, —S(O)₂OR¹, —OC(O)R¹, —OC(O)OR¹, —OC(O)N(R¹)₂, —N(R¹)C(O)R¹, —N(R¹)C(O)OR¹, —N(R¹)C(O)N(R¹)₂, C₃-C₈ cycloalkyl, heterocyclyl, aryl, or heteroaryl, wherein each R¹ is independently hydrogen, C₁-C₆ alkyl, or C₁-C₆ haloalkyl.

In an embodiment of any of the preceding embodiments of formula (I), at least one R group, when present is electrically similar to hydrogen and the remaining R groups are each independently halogen, cyano, nitro, C₁-C₄ alkyl, C₁-C₄ haloalkyl, —OR¹, —N(R¹)₂, —SR¹, —C(O)OR¹, —C(O)R¹, —C(O)N(R¹)₂, —S(O)₂R¹, —S(O)₂N(R¹)₂, or —S(O)₂OR¹, wherein each R¹ is independently hydrogen, C₁-C₆ alkyl, or C₁-C₆ haloalkyl.

In an embodiment of any of the preceding embodiments of formula (I), at least one R group, when present is C₁₋₆ alkyl (e.g., methyl or tert-butyl), and the remaining R groups are each independently halogen, cyano, nitro, C₁-C₄ alkyl, C₁-C₄ haloalkyl, —OR¹, —N(R¹)₂, —SR¹, —C(O)OR¹, —C(O)R¹, —C(O)N(R¹)₂, —S(O)₂R¹, —S(O)₂N(R¹)₂, or —S(O)₂OR¹, wherein each R¹ is independently hydrogen, C₁-C₆ alkyl, or C₁-C₆ haloalkyl.

In an embodiment of any of the preceding embodiments of formula (I), wherein each m is independently 0, 1, or 2; at least one R group, when present is electrically similar to hydrogen; and the remaining R groups are each independently halogen, cyano, nitro, trifluoromethyl, C(O)OR¹, —C(O)R¹, —C(O)N(R¹)₂, —S(O)₂R¹, —S(O)₂N(R¹)₂, —S(O)₂OR¹.

In an embodiment of any of the preceding embodiments of formula (I), wherein each m is independently 0, 1, or 2; at least one R group, when present is C₁₋₆ alkyl (e.g., methyl or tert-butyl); and the remaining R groups are each independently halogen, cyano, nitro, trifluoromethyl, C(O)OR¹, —C(O)R¹, —C(O)N(R¹)₂, —S(O)₂R¹, —S(O)₂N(R¹)₂, —S(O)₂OR¹.

In an embodiment of any of the preceding embodiments of formula (I), each m is independently 0, 1, or 2; at least one R group, when present is electrically similar to hydrogen; and the remaining R groups are each independently C₁-C₆ alkyl, —OR¹, —N(R¹)₂, or —SR¹.

In an embodiment of any of the preceding embodiments of formula (I), each m is independently 0, 1, or 2; at least one R group, when present is C₁₋₆ alkyl (e.g., methyl or tert-butyl); and the remaining R groups are each independently C₁-C₆ alkyl, —OR¹, —N(R¹)₂, or —SR¹.

In an embodiment of any of the preceding embodiments of formula (I), when n is 2 or greater, then the sets of R groups on each phenyl ring in the compound of formula (I) are not identical.

In an embodiment of any of the preceding embodiments of formula (I), when n is 2 or greater, then at least one of the sets of R groups on each phenyl ring in the compound of formula (I) is not identical to the sets of R groups on the remaining phenyl groups.

In an embodiment of any of the preceding embodiments of formula (I), when n is 3 or greater, then the sets of R groups on at least two phenyl rings in the compound of formula (I) are not identical.

In an embodiment of any of the preceding embodiments of formula (I), when n is 2 or greater, the sets of substituents (i.e., including hydrogens and R groups) on each phenyl group are electrically similar to one another, but the total mass of the substituents for at least one of the phenyl groups is not identical to the other phenyl groups.

In an embodiment of any of the preceding embodiments of formula (I), n is 1-90, or 1-80, or 1-70, or 1-60, or 1-50, or 1-40, or 1-30. In certain embodiments, n is 1-25, or 1-20, or 1-15, or 1-10. In certain other embodiments, n is 1-9, or 1-8, or 1-7, or 1-6, or 1-5, or 1-4, or 1-3, or 1-2.

In an embodiment of any of the preceding embodiments of formula (I), n is 2-100. In certain embodiments, n is 2-90, or 2-80, or 2-70, or 2-60, or 2-50, or 2-40, or 2-30. In certain embodiments, n is 2-25, or 2-20, or 2-15, or 2-10. In certain other embodiments, n is 2-9, or 2-8, or 2-7, or 2-6, or 2-5, or 2-4, or 2-3.

In yet other embodiments, n is 1. In yet other embodiments, n is 2. In yet other embodiments, n is 3. In yet other embodiments, n is 4. In yet other embodiments, n is 5. In yet other embodiments, n is 6. In yet other embodiments, n is 7. In yet other embodiments, n is 8. In yet other embodiments, n is 9. In yet other embodiments, n is 10.

In one particular embodiment of any of the preceding embodiments of formula (I), the compound is according to formula (II),

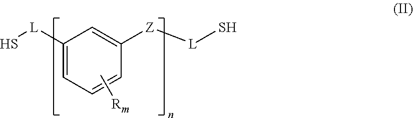

wherein

n is 1-100;

Z is a bond or —O—;

each m is independently 0 or 1;

each R is independently an electron-donating group, an electron-withdrawing group, or a group electrically similar to hydrogen; (e.g., each R is independently halogen, cyano, nitro, C₁-C₄ alkyl, C₁-C₄ haloalkyl, —OR¹, —N(R¹)₂, —SR¹, —C(O)OR¹, —C(O)R¹, —C(O)N(R¹)₂, —S(O)₂R¹, —S(O)₂N(R¹)₂, or —S(O)₂OR¹, wherein each R¹ is independently hydrogen, C₁-C₆ alkyl, or C₁-C₆ haloalkyl);

and

each L is independently a bond or a divalent linking group.

Preferred embodiments for N, Z, m, R, and L are as described above for compounds of formula (I).

Assemblies of any of the preceding compounds of formula (I), (II), and any embodiment thereof, between a first surface and a second surface may be prepared according to methods known in the art, wherein for each molecule bridging the first surface and second surface, one R^{E} group of the molecule is chemically bonded or associated with the first surface, and the second R^{E} group of the molecule is chemically bonded or associated with the second surface.

A self-assembled monolayer (SAM) may be prepared on the first surface according to methods known in the art, such as vapor deposition or deposition by immersion of the surface in a solution of the compound of formula (I) or (II). For example, see, Love et al., Chem. Rev. 2005, 105, 1103-1169, which is incorporated by reference in its entirety. The second layer may be deposited over the (SAM) to complete the assembly. For example, where the second layer is a metal layer, the second layer can be pressed mechanically into contact (e.g. a thin metal foil), deposited over the SAM by chemical vapor deposition, metal evaporation, or electroless deposition methods known to one skilled in the art. For example, compounds including thiol end groups (—SH) are known to associate with metal surfaces, such as silver, gold, and copper surfaces. Thiols can coat the surface at a concentration of about 0.1 mM to about 10 mM; or about 0.5 mM to about 10 mM; or about 1 mM to about 10 mM; or about 1 mM to about 5 mM; or about 1 mM concentration.

Examples of surfaces and suitable end groups for forming an assembly thereon include, but are not limited to:

| R^{E} | Surface |
|---|---|
| —OH | FeₓO_{y}, Si |
| —COOH | Al₂O₃, FeₓO_{y}, Ni, Ti, TiO₂ |
| —CN | Ag, Au |
| —NH₂ | FeS₂, Stainless steel, CdSe, YBa₂Cu₃O₇, mica |
| —N≡N⁺ | Pd, Si, GaAs |
| —SH | Ag, Ag_{0.90}Ni_{0.10}, AgS, Au, AuAg, AuCu, AuPd, Au, CdTe, CdSe, CdS, Cd, |
| | FePt, GaAs, Ge, Hg, HgTe, InP, Ir, Ni, PbS, Pd, PdAg, Pt, Ru, YBa₂Cu₃O₇, |
| | Zn, ZnSe, ZnS, stainless steel |
| —S₂O₃⁻Na⁺ | Au, Cu |
| —SAc | Au |
| 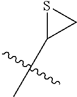 | Au |
| —SR′ | Au |
| —SSR′ | Ag, Au, CdS, Pd |
| 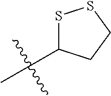 | Au |
| —C(S)SH | Au, CdSe |
| —SeH | Ag, Au, CdS, CdSe |
| —SeSeR′ | Au |
| —PR′₂ | Au, FeS₂, CdS, CdSe, CdTe |
| —P(O)R′₂ | Co, CdS, CdSe, CdTe |
| —P(O)(O)₂2-, | Al, Al—OH, Ca₁₀(PO₄CO₃)₆(OH)₂, GaAs, GaN, indium-tin-oxide, mica, TiO₂, |
| —P(O)(OH)₂ | ZrO₂, CdSe, CdTe, |
| —PO₄2- | Al₂O₃, Nb₂O₅, Ta₂O₃, TiO₂ |
| —N≡C | Pt |
| —C(H)═CH₂, | Si |
| —C≡CH, | |
| —SiX₃ (X = | HfO₂, ITO, PtO, TiO₂, ZrO₂ |
| H, Cl, OR′) | |

In certain embodiments, each of the first and second surfaces is a metal surface. For example, each of the first and second surfaces is independently Al, Ag, Au, Ni, Ti, Pd, Pt, Ag_{0.90}Ni_{0.10}, AuAg, AuCu, AuPd, Cd, FePt, Ir, PdAg, Ru, stainless steel, or Zn. In certain embodiments, each of the first and second surfaces is a metal surface. For example, each of the first and second surfaces is independently Al, Ag, Au, Ni, Ti, Pd, Pt, Cd, Ir, Ru, or Zn. In other examples, each metal surface is a gold, silver, copper, platinum, palladium, aluminum, or titanium surface.

In one embodiment, each metal surface is a gold, silver, or copper surface and each R^{E} is —SH. In another embodiment, each metal surface is a gold, silver, platinum, or copper surface and each R^{E} is a bond.

In another embodiment of the assemblies herein, each R^{E} is independently a bond, —SH, —COOH, —P(O)(OH)₂, —Si(OR¹)₃₋ₐ(OH)ₐ, or —C≡N wherein a is 1, 2, or 3.

### Methods for Preparation of the Compounds

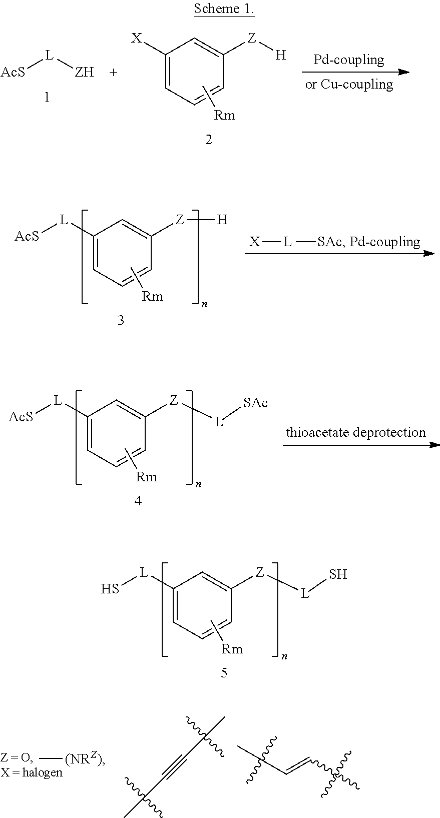

For example, compounds described herein where R^{E} is —SH may be prepared according to Scheme 1. A compound of generic formula (I) having an acetate protected thiol group linked through a linking group (L) to a —ZH group, each as defined herein, may be coupled under palladium-catalyzed conditions familiar to those skilled in the art to a compound of formula (2) having a halogen (X, e.g., bromo or iodo) and ZH group in a meta-relationship to one another. When Z is a vinylene or ethynylene group, the palladium-catalyzed conditions may comprise Heck or Sonogashira coupling conditions comprising a catalyst, such as, but not limited to, Ph(PPh₃)₄, Pd₂(dibenzylideneacetone)₃ [i.e., Pd₂(dba)₃], PdCl₂, Pd(PPh₃)₂Cl₂, or Pd(OAc)₂, and a copper source, such as CuI, in the presence of a base, such as, cesium carbonate, potassium carbonate, N,N-diisopropylamine, tributylamine, or triethylamine. When Pd₂(dba)₃, PdCl₂, Pd(PPh₃)₂Cl₂, or Pd(OAc)₂ is used, a phosphine such as triphenyphospine, tri(tert-butyl)phosphine, or tri(cyclohexyl)phosphine may also be used. Such couplings may give the compounds of general formula (3) where n may be controlled by the ratio of end group containing compound (1) to compound (2) present under the reaction conditions as is familiar to those skilled in the art. The second terminal linking group having an acetate protected thiol may be attached to the compound of formula (3) under similar palladium catalyzed conditions as the preceding step using a compound of the formula X-L-SAc to generate compound (4). Finally, the thioacetate groups may be deprotected to yield the free thiol groups of compound (5), for example, by hydrolysis with ammonium hydroxide, propylamine, 1,8-diazabicyclo[5.4.0]undec-7-ene (DBU), hydrochloric acid sodium hydroxide, sodium methoxide, potassium hydroxide, or potassium carbonate.

Where Z is —O— or —S— in preparation of compound (3) Ullmann-type copper-catalyzed reaction conditions may be used. For example, compounds (1) and (2) may be coupled in the presence of CuI, a chelating bidentate base, such as 2,2′-bipyridine, 1,10-phenanthroline or a substituted 2,2′-bipyridine or 1,10-phenanthroline (e.g., neocuproine, 3,4,7,8-tetramethyl-1,10-phenanthroline), and a base such as potassium phosphate, cesium carbonate, potassium carbonate, or sodium t-butoxide. Such couplings may give the compounds of general formula (3) where n may be controlled by the ratio of end group containing compound (1) to compound (2) present under the reaction conditions as is familiar to those skilled in the art. The second terminal linking group having an acetate protected thiol may be attached to the compound of formula (3) under similar palladium catalyzed conditions as the preceding step using a compound of the formula X-L-SAc to generate compound (4), and the thioacetate groups may be deprotected to yield the free thiol groups of compound (5).

Where Z is —O— or —N(R^{Z})—, in preparation of compound (3), palladium-catalyzed reaction conditions may be used. For example, compounds (1) and (2) may be coupled in the presence of Ph(PPh₃)₄, Pd₂(dba)₃, PdCl₂, Pd(PPh₃)₂Cl₂, or Pd(OAc)₂, a base such as potassium phosphate, sodium t-butoxide, cesium carbonate, or potassium carbonate, and a bulky phosphine ligand, such as, but not limited to, 2-Dicyclohexylphosphino-2′,6′-dimethoxybiphenyl,_{—}2-Dicyclohexylphosphino-2′,4′,6′-triisopropylbiphenyl, 2-Dicyclohexylphosphino-2′-methylbiphenyl,_{—}2-Di-tert-butylphosphino-2′,4′,6′-triisopropylbiphenyl,_{—}2-Di-tert-butylphosphino-3,4,5,6-tetramethyl-2′,4′,6′-triisopropyl-1,1′-biphenyl,_{—}2-Di-tert-butylphosphino-2′-methylbiphenyl, 2-Di-tert-butylphosphino-2′-(N,N-dimethylamino)biphenyl, 2-Dicyclohexylphosphino-2′-(N,N-dimethylamino)biphenyl, 2-(Dicyclohexylphosphino)biphenyl, _{—}2-(Di-tert-butylphosphino)biphenyl, 2-Dicyclohexylphosphino-2′,6′-diisopropoxybiphenyl_{—}, 2-Diphenylphosphino-2′-(N,N-dimethylamino)biphenyl, and sodium 2-dicyclohexylphosphino-2′,6′-dimethoxybiphenyl-3′-sulfonate. See, for example, U.S. Pat. Nos. 6,762,329; 6,166,226; 6,235,871; and 6,307,087, each of which are hereby incorporated by reference. The second terminal linking group to an acetate protected thiol may be attached to the compound of formula (3) under similar palladium catalyzed conditions as the preceding step using a compound of the formula X-L-SAc to generate compound (4). Finally, the thioacetate groups may be deprotected to yield the free thiol groups of compound (5).

In the preceding methods, compound (3) may be formed a polydisperse mixture of compounds having n values as defined herein. Such may be separated according to methods known in the art, such as column chromatography or high-performance liquid chromatography using a size-exclusion column either before or after preparation of compounds (3), (4) and/or (5).

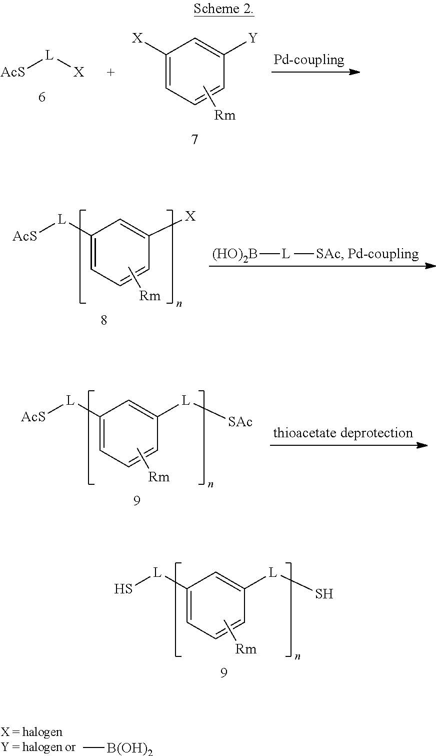

In another example, compounds described herein where R^{E} is —SH and Z is a bond (e.g., m-polyphenylenes) may be prepared according to Scheme 2. A compound of generic formula (6) having an acetate protected thiol group linked to a halogen, where L is a linking group as defined herein may be coupled under either palladium-catalyzed conditions familiar to those skilled in the art with a compound of formula (7) having a halogen (X e.g., bromo or iodo) and a Y group in a meta-relationship to one another. Under palladium catalyzed conditions (e.g., Suzuki coupling conditions), Y is a boronic acid (—B(OH₂)) and the reaction is performed in the presence of a palladium catalyst such as Ph(PPh₃)₄, Pd₂(dba)₃, or Pd(OAc)₂, and a base such as potassium carbonate, potassium phosphate, or potassium t-butoxide. When Pd₂(dba)₃ or Pd(OAc)₂ is used, a phosphine such as triphenyphospine, tri(tert-butyl)phosphine, di(t-butyl)methylphosphine, or tri(cyclohexyl)phosphine may be used. Such couplings may give the compounds of general formula (8) where n may be controlled by the ratio of end group containing compound (6) to compound (7) present under the reaction conditions as is familiar to those skilled in the art.

The second terminal linking group having an acetate protected thiol may be attached to the compound of formula (8) under similar palladium catalyzed conditions as the preceding step using a compound of the formula (HO)₂B-L-SAc to generate compound (9). Finally, the thioacetate groups may be deprotected to yield the free thiol groups of compound (10). Compound (8) may be formed as a polydisperse mixture of compounds having n values as defined herein. Such may be separated according to methods known in the art, such as column chromatography or high-performance liquid chromatography using a size-exclusion column either before or after preparation of compounds (8), (9) and/or (10).

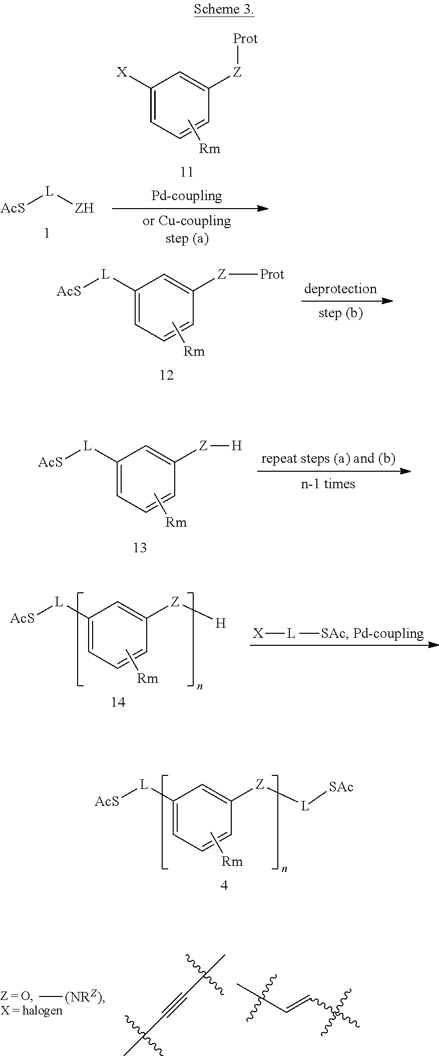

Alternatively, compounds described herein where R^{E} is —SH may be prepared according to the iterative process of Scheme 3. Therein, compound (1) is coupled to compound (11) under the palladium- or copper-catalyzed conditions described above for Scheme 1 (“step a”) to yield compound (12). Compound (11) comprises a halogen (X) and a protected “ZH” group (—Z-Prot) in a meta-relationship. Examples of suitable protecting groups include, but are not limited to, trimethylsilyl, t-butyldimethylsilyl, acetyl, and the like. The protecting group is removed in “step (b)” from compound (12), under conditions known to those skilled in the art (e.g., for trimethylsilyl groups, treatment of compound (12) with tetrabutylammonium fluoride) to yield the free —ZH functional group of compound (13). Steps (a) and (b) may be repeated iteratively to yield compound (14) having the desired value of n. Finally, compound (14) may be coupled to a compound of the formula X-L-SAc, to yield compound (4), which may be deprotected to yield compound (5), each as described above.

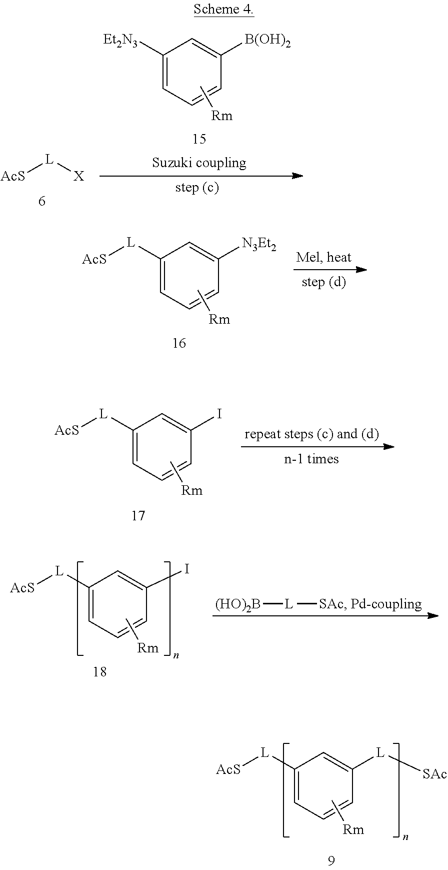

Similarly, compounds described herein where R^{E} is —SH and Z is a bond may be prepared according to the iterative process of Scheme 4. Therein, compound (6) is coupled to compound (15) under the Suzuki coupling conditions described above for Scheme 2 (“step c”) to yield compound (16). Compound (15) comprises a boronic acid and a “protected” halogen group in the form of a diethyltriazene group in a meta-relationship. Such diethyltriazene groups may be prepared from the corresponding amine by treatment with NaNO₂ and HCl to generate a diazonium which is treated with diethylamine and potassium carbonate. The protecting group is removed in “step (d)” from compound (16), under conditions known to those skilled in the art (e.g., heating in the presence of methyl iodide) to yield the iodo-functionalized compound (17). Steps (c) and (d) may be repeated iteratively to yield compound (18) having the desired value of n. Finally, compound (18) may be coupled to a compound of the formula (HO)₂B-L-SAc, to yield compound (9), which may be deprotected to yield compound (10), each as described above.

The preceding methods may be readily modified by one skilled in the art to generate the compounds of formulas (I) and (II) described herein having R^{E} groups other than thiols. For example, suitably protected amine groups may replace the -Sac groups in the above methods for generating compounds where R^{E} is amino

### DEFINITIONS

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆alkoxycarbonyloxy and —OC(O)C₁-C₆alkyl indicate the same functionality; similarly arylalkyl and -alkylaryl indicate the same functionality.

Further, certain monovalent terms herein, such as alkyl, aryl, heteroaryl, cycloalkyl, and heterocyclyl are also used as divalent terms, for example within —C₀-C₁₀ alkyl-J-. In such cases, it is understood that one skilled in the art would interpret such use as a divalent radical bridging two parent moities.

The term “alkyl” as used herein, means a straight or branched chain hydrocarbon containing from 1 to 10 carbon atoms, unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂⁻, —CH₂CH₂CHC(CH₃)—, —CH₂CH(CH₂CH₃)CH₂—.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The terms “cyano” and “nitrile” as used herein, mean a —CN group.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

“Cycloalkenyl” as used herein refers to a monocyclic or a bicyclic cycloalkenyl ring system. Monocyclic ring systems are cyclic hydrocarbon groups containing from 3 to 8 carbon atoms, where such groups are unsaturated (i.e., containing at least one annular carbon-carbon double bond), but not aromatic. Examples of monocyclic ring systems include cyclopentenyl and cyclohexenyl. Bicyclic cycloalkenyl rings are bridged monocyclic rings or a fused bicyclic rings. Bridged monocyclic rings contain a monocyclic cycloalkenyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic cycloalkenyls include, but are not limited to, norbornenyl and bicyclo[2.2.2]oct-2-enyl. Fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. The bridged or fused bicyclic cycloalkenyl is attached to the parent molecular moiety through any carbon atom contained within the monocyclic cycloalkenyl ring. Cycloalkenyl groups are optionally substituted with one or two groups which are independently oxo or thia.

The term “halo” or “halogen” as used herein, means —Cl, —Br, —I or —F.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a phenyl ring or a monocyclic heteroaryl, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, and purinyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The term “heterocyclyl” as used herein, means a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl(thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl.

The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “nitro” as used herein, means a —NO₂ group.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “thia” as used herein means a ═S group.

The term “unsaturated” as used herein means the referenced chemical structure contains at least one multiple carbon-carbon bond, but is not aromatic. For example, an unsaturated cycloalkyl group as defined herein includes cyclohexenyl, cyclopentenyl, cyclohexadienyl, and the like.

### 3. CONCLUSION

An exemplary embodiment of the present invention has been described above. Those skilled in the art will understand, however, that changes and modifications may be made to this embodiment without departing from the true scope and spirit of the invention, which is defined by the claims.

What is claimed:

1. A thermoelectric device, comprising:
a first electrode;
a second electrode;
an electrical transmission medium electrically connected to the first and second electrodes, wherein the electrical transmission medium comprises a quantum conductor that exhibits at least one transmission node or a transmission resonance due to quantum interference.

2. The thermoelectric device of claim 1, wherein the thermoelectric device is configured to be operable as a thermoelectric power generator.

3. The thermoelectric device of claim 2, wherein the thermoelectric device is configured to develop a voltage difference between the first and second electrodes in response to a temperature difference between the first and second electrodes.

4. The thermoelectric device of claim 3, wherein the first electrode is in thermal contact with a heat source and the second electrode is in thermal contact with an ambient, such that the first electrode is at a higher temperature than the second electrode.

5. The thermoelectric device of claim 1, wherein the thermoelectric device is operable as a Peltier cooler, the Peltier cooler having a low-temperature side and a high-temperature side.

6. The thermoelectric device of claim 5, wherein the thermoelectric device is configured to transfer heat from the low-temperature side to the high-temperature side in response to an applied voltage between the first and second electrodes.

7. The thermoelectric device of claim 1, wherein the quantum conductor comprises an organic molecule bonded to the first and second electrodes.

8. The thermoelectric device of claim 7, wherein the organic molecule comprises a plurality of meta-connected benzene rings.

9. The thermoelectric device of claim 7, wherein the electrical transmission medium comprises a plurality of the organic molecules bonded to the first and second electrodes.

10. The thermoelectric device of claim 9, wherein the organic molecules are arranged in at least one self-assembled monolayer (SAM).

11. The thermoelectric device of claim 1, further comprising a third electrode electrically connected to the electrical transmission medium.

12. The thermoelectric device of claim 11, wherein the electrical transmission medium comprises a first quantum conductor between the first and second electrodes and a second quantum conductor between the second and third electrodes.

13. The thermoelectric device of claim 12, wherein the first quantum conductor comprises an N-type organic molecule and the second quantum conductor comprises a P-type organic molecule.

14. The thermoelectric device of claim 13, wherein the N-type organic molecule is bonded to the first and second electrodes and the P-type organic molecule is bonded to the second and third electrodes.

15. The thermoelectric device of claim 14, wherein the electrical transmission medium comprises a plurality of N-type organic molecules bonded to the first and second electrodes and a plurality of P-type organic molecules bonded to the second and third electrodes.

16. The thermoelectric device of claim 15, wherein the N-type organic molecules and P-type organic molecules are arranged in self-assembled monolayers (SAMs).

17. The thermoelectric device of claim 7, wherein the organic molecules are of the formula, 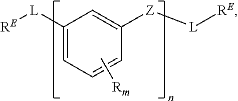
wherein
n is 1-100;
Z is a bond, —O—, —S—, —N(R^{Z})—, —C(O)—, —S(O)—, —S(O)₂—, —C(R^{Z})₂—, —C(R^{Z})═C(R^{Z})—, —C≡C—, wherein each R^{Z} is independently hydrogen or C₁-C₆ alkyl;
each m is independently 0, 1, 2, 3, or 4;
each R is independently an electron-donating group, an electron-withdrawing group, or a group electrically similar to hydrogen;
each L is independently a bond or a divalent linking group;
each R^{E} is independently a functional group capable of bonding to or associating with a metal surface;
wherein
(i) when the organic molecule is an N-type organic molecule, then at least one R group is independently C₁-C₆ alkyl, —OR¹, —N(R¹)₂, or —SR¹; and
(ii) when the organic molecule is a P-type organic molecule, then each at least one R group is independently halogen, cyano, nitro, trifluoromethyl, C(O)OR¹, —C(O)R¹, —C(O)N(R¹)₂, —S(O)₂R¹, —S(O)₂N(R¹)₂, —S(O)₂OR¹.

18.-26. (canceled)

27. The thermoelectric device of claim 17, wherein each L is independently of the formula, —(C₀-C₁₀ alkyl-J)₀₋₂—, wherein each J is independently a bond, aryl, heteroaryl, C₃-C₈ cycloalkyl, or heterocyclyl; and no more than one methylene in each alkyl group is optionally and independently replaced by —O—, —S—, —N(R⁰)—, —C(H)═C(H)—, —C≡C—, —C(O)—, —S(O)—, —S(O)₂—, —P(O)(OH)—, —OP(O)(OH)—, —P(O)(OH)O—, —N(R⁰)P(O)(OH)—, —P(O)(OH)N(R⁰)—, —OP(O)(OH)O—, —OP(O)(OH)N(R⁰)—, —N(R⁰)P(O)(OH)O—, —N(R⁰)P(O)(OH)N(R⁰)—, —C(O)O—, —C(O)N(R⁰)—, —OC(O)—, —N(R⁰)C(O)—, —S(O)O—, —OS(O)—, —S(O)N(R⁰)—, —N(R⁰⁰)S(O)—, —S(O)₂O—, —OS(O)₂—, —S(O)₂N(R⁰)—, —N(R⁰)S(O)₂—, OC(O)O—, —OC(O)N(R⁰)—, —N(R⁰)C(O)O—, —N(R⁰)C(O)N(R⁰)—, —OS(O)O—, —OS(O)N(R⁰)—, —N(R⁰)S(O)O—, —N(R⁰)S(O)N(R⁰)—, —OS(O)₂O—, —OS(O)₂N(R⁰)—, —N(R⁰)S(O)₂O—, or —N(R⁰)S(O)₂N(R⁰)—, wherein each R⁰ is independently hydrogen or C₁-C₆ alkyl.

28.-29. (canceled)

30. The thermoelectric device of claim 17, wherein each L is independently of the formula, 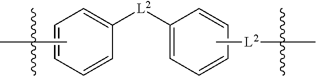
wherein each L² is independently a bond, —CH₂—, —O—, —S—, —N(R⁰)—, —C(H)═C(H)—, —C≡C—, —C(O)—, —S(O)₂—, —C(O)O—, —C(O)N(R⁰)—, —OC(O)—, —N(R⁰)C(O)—, —S(O)₂N(R⁰)—, —N(R⁰)S(O)₂—, OC(O)O—, —OC(O)N(R⁰)—, —N(R⁰)C(O)O—, or —N(R⁰)C(O)N(R⁰—.

31.-36. (canceled)

37. The thermoelectric device of claim 17, wherein when n is 2 or greater, then the sets of R groups on each phenyl ring in the compound of formula (I) are not identical.

38.-44. (canceled)

45. The thermoelectric device of claim 17 of the formula 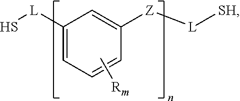
wherein
n is 1-100;
Z is a bond or —O—;
each m is independently 0 or 1;
each R is independently an electron-donating group, an electron-withdrawing group, or a group electrically similar to hydrogen;
and
each L is independently a bond or a divalent linking group.

46. The thermoelectric device of claim 11, wherein the thermoelectric device is operable as a thermoelectric power generator.

47. The thermoelectric device of claim 46, wherein the first and third electrodes are on a first side of the thermoelectric device and the second electrode is on a second side of the thermoelectric device.

48. The thermoelectric device of claim 47, wherein the thermoelectric device is configured to develop a voltage difference between the first and third electrodes in response to a temperature difference between the first side and the second side.

49. The thermoelectric device of claim 48, wherein the second side is in thermal contact with a heat source and the first side is in thermal contact with an ambient, such that the first and third electrodes are at a lower temperature than the second electrode.

50.-52. (canceled)

53. A thermoelectric power generator for generating a voltage difference between a first electrical contact and a second electrical contact in response to a temperature difference between a first heat-transfer surface and a second heat-transfer surface, the thermoelectric power generator comprising:
at least one N-type thermoelectric structure comprising N-type organic molecules arranged in a self-assembled monolayer; and
at least one P-type thermoelectric structure comprising P-type organic molecules arranged in a self-assembled monolayer,
wherein the at least one N-type thermoelectric structure and the at least one P-type thermoelectric structure are electrically connected in series between the first and second electrical contacts and thermally connected in parallel between the first and second heat-transfer surfaces.

54. The thermoelectric power generator of claim 53, wherein the N-type organic molecules individually comprise electron donor substituents on a backbone of meta-connected benzene rings and the P-type organic molecules individually comprise electron acceptor substituents on a backbone of meta-connected benzene rings.

55. A Peltier cooler for transferring heat from a low-temperature surface to a high-temperature surface in response to an applied voltage between a first electrical contact and a second electrical contact, the Peltier cooler comprising:
at least one N-type thermoelectric structure comprising N-type organic molecules arranged in a self-assembled monolayer; and
at least one P-type thermoelectric structure comprising P-type organic molecules arranged in a self-assembled monolayer,
wherein the at least one N-type thermoelectric structure and the at least one P-type thermoelectric structure are electrically connected in series between the first and second electrical contacts and thermally connected in parallel between the low-temperature and high-temperature surfaces.

56. The Peltier cooler of claim 55, wherein the N-type organic molecules individually comprise electron donor substituents on a backbone of meta-connected benzene rings and the P-type organic molecules individually comprise electron acceptor substituents on a backbone of meta-connected benzene rings.

57. A compound of the formula, 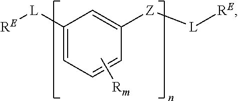
wherein
n is 1-100;
Z is a bond, —O—, —S—, —N(R^{Z})—, —C(O)—, —S(O)—, —S(O)₂—, —C(R^{Z})₂—, —C(R^{Z})═C(R^{Z})—, —C≡C—, wherein each R^{Z} is independently hydrogen or C₁-C₆ alkyl;
each m is independently 0, 1, 2, 3, or 4;
each R is independently an electron-donating group, an electron-withdrawing group, or a group electrically similar to hydrogen;
each L is independently a bond or a divalent linking group;
each R^{E} is independently a functional group capable of bonding to or associating with a metal surface.

58.-85. (canceled)

86. An assembly comprising a first metal surface; a second metal surface; and one or more molecules bridging the first and second metal surfaces, wherein
each molecule is of the formula 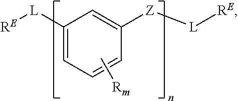
wherein
n is 1-100;
Z is a bond, —O—, —S—, —N(R^{Z})—, —C(O)—, —S(O)—, —S(O)₂—, —C(R^{Z})₂—, —C(R^{Z})═C(R^{Z})—, —C≡C—, wherein each R^{Z} is independently hydrogen or C₁-C₆ alkyl;
each m is independently 0, 1, 2, 3, or 4;
each R is independently an electron-donating group, an electron-withdrawing group, or a group electrically similar to hydrogen;
each L is independently a bond or a divalent linking group;
each R^{E} is independently a functional group capable of bonding to or associating with the first metal surface or second metal surface;
and wherein for each molecule bridging the first metal surface and second metal surface, one R^{E} group of the molecule is chemically bonded or associated with the first metal surface, and the second R^{E} group of the molecule is chemically bonded or associated with the second metal surface.

87.-93. (canceled)
